SEQUENCE LISTING (3 sequences)

SEQ ID NO: 1 - DNA, 48 bp, Artificial Sequence
  DNA encoding shC RNA
cctaaggttaagtcgccctcgctcgagcgagggcgacttaaccttagg

SEQ ID NO: 2 - DNA, 48 bp, Artificial Sequence
  DNA encoding Sh1 RNA
gccagaatatcattagcgtttctcgagaaacgctaatgatattctggc

SEQ ID NO: 3 - DNA, 48 bp, Artificial Sequence
  DNA encoding sh2 RNA
gcgcatgacaataagcagattctcgagaatctgcttattgtcatgcgc

(19) United States
(12) Patent Application Publication
(10) Pub. No.: US 2018/0209979 A1
(43) Pub. Date: Jul. 26, 2018

(54) METHOD FOR INDIVIDUALIZED CANCER THERAPY

(71) Applicants: INSERM (INSTITUT NATIONAL DE LA SANTE ET DE LA RECHERCHE), Paris (JP); CENTRE NATIONAL DE LA RECHERCHE SCIENTIFIQUE, Paris (JP); UNIVERSITE DE NICE SOPHIA ANTIPOLIS, Nice (FR); CENTRE SCIENTIFIQUE DE MONACO, Monaco (MC)

(72) Inventors: Vincent PICCO, Monaco (MC); Gilles Page, Nice (FR)

(21) Appl. No.: 15/745,341

(22) PCT Filed: Jul. 13, 2016
(86) PCT No.: PCT/EP2016/066624

§ 371 Date: Jan. 16, 2018

(30) Foreign Application Priority Data
Jul. 17, 2015 (EP) 15306173.4

(51) Int. Cl. G01N 33/574; C12N 15/113; C12N 15/115; A61K 31/713; A61P 35/00; A61K 45/06

(52) U.S. Cl. CPC G01N 33/57407; C12N 15/113; C12N 15/115; A61K 31/713; C12N 2310/16; A61K 45/06; C12N 2310/11; C12N 2310/141; A61P 35/00

(57) ABSTRACT

An in vitro method for predicting a likelihood of an individual having a cancer to efficiently respond to an anti-cancer treatment, said method including the steps: of a) measuring the level of nuclear expression of TRF2 in a biopsy obtained from said individual, b) comparing the level obtained in step a) to a reference value, and c) determining the predicted likelihood of said individual to efficiently respond to said anti-cancer treatment from the comparison performed in step b).

### DOMAIN OF THE INVENTION

The present invention provides a method for individualized cancer therapy.

More particularly, the present invention provides a method for predicting the responsiveness of an individual having an oral squamous cell carcinoma (OSCC) towards one or more epidermal growth factor receptor (EGFR) inhibitors, as chemotherapy agents, prior to treating said individual with such chemotherapy agents.

### BACKGROUND OF THE INVENTION

Head and neck cancer is the fifth most common cancer in France and 90% of them are Oral Squamous Cell Carcinomas (OSCC), an aggressive malignancy arising from the epithelial cells of oral mucosa. 35% of these tumors are located in the mouth, often associated with a tobacco and/or alcohol intoxication. OSCC is the sixth most common type of neoplasm worldwide. The diagnosis is confirmed by pathological examination of a biopsy.

Oral cancer is often diagnosed when the cancer has metastasized to another location, in general in the lymph nodes of the neck. Prognosis at this stage of discovery is significantly worse than when it is caught at an early stage, i.e. in a localized intra oral area. At a later stage, metastasis may be often accompanied with an invasion of the primary tumor into deep local tissues.

Oral cancer is particularly insidious because in its early stages it may not be noticed by the patient, since it may progress without producing significant pain or symptoms. Therefore, their occurrence significantly increases the risk of producing subsequent primary tumors. This means that individuals surviving a first encounter with the disease, have up to a 20 times higher risk of developing a second cancer.

Oral Squamous Cell Carcinomas (OSCC) may be featured by the appearance of white or red patches of tissue in the mouth, or small indurated ulcers, similar to common canker sores. Other symptoms may include; a lump or mass which can be felt inside the mouth or neck, pain or difficulty in swallowing, speaking, or chewing, any wart like masses, hoarseness which lasts for a long time, or any numbness in the oral/facial region. Unilateral persistent ear ache can also be a warning sign.

The actual curative treatment of OSCC often relies upon a multidisciplinary approach, usually combining chemotherapy with concurrent radiation, and sometimes with surgery. Despite the development of targeted therapies, this cancer type still remains incurable at the metastatic grade. However existing treatments, such as invasive head and neck surgery, radiotherapy and chemotherapy, generate aesthetic and functional sequels that have a negative impact on quality of life.

The overall survival ranges from 12 to 50% at 5 years depending on the localization in the mouth. 80% of OSCC are associated with over-expression and activation of epidermal growth factor receptor (EGFR) and mitogen-activated protein kinase (MAPK) signaling pathways.

Therefore, efforts need to be intensified to identify new therapeutic strategies for a better quality of life and perhaps at term to prolong survival. The OSCC patients undergo surgery, then radiotherapy associated or not with chemotherapy. The major debilitating side effects of radio/chemo are mucitis. Therefore, it is important to identify individuals with a poor prognosis in order to intensify the treatment and administrate a less aggressive individualized treatment, most suitable for ameliorating the overall survival of said individuals.

### SUMMARY OF THE INVENTION

In one aspect, the invention relates to an in vitro method for predicting a likelihood of an individual having a cancer to efficiently respond to an anti-cancer treatment, said method comprising the steps of:

a) measuring the level of nuclear expression of TRF2 in a biopsy obtained from said individual,

b) comparing the level obtained in step a) to a reference value, and

c) determining the predicted likelihood of said individual to efficiently respond to said anti-cancer treatment from the comparison performed in step b).

In a specific aspect of the invention, said cancer is an oral squamous cell carcinoma (OSCC).

In another aspect, the invention relates to an in vitro method for predicting an outcome for an individual having an oral squamous cell carcinoma, said method comprising the steps of:

a) measuring a level of nuclear expression of TRF2 in a biopsy obtained from said individual,

b) comparing a level obtained in step (a) to a reference value, and

c) determining a prognostic of said individual from said comparison performed in step b).

In a still another aspect, the invention also relates to an inhibitor of the TRF2 gene expression for use in the improvement of the treatment of an individual having an oral squamous cell carcinoma.

In one aspect, the invention relates to a kit for use in the improvement of the treatment of an individual having an oral squamous cell carcinoma comprising:

an inhibitor of the TRF2 gene expression in a physiologically acceptable excipient, and,

an anti-cancer compound in a physiologically acceptable excipient.

In another aspect, the invention relates to an ex vivo or in vitro use of a level of nuclear expression of TRF2 as a biomarker for predicting an outcome for an individual having an oral squamous cell carcinoma.

In another aspect, the invention relates to an ex vivo or in vitro use of a level of nuclear expression of TRF2 as a biomarker for predicting a likelihood of an individual having an oral squamous cell carcinoma to efficiently respond to an anti-cancer treatment.

In another aspect, the invention relates to an ex vivo or in vitro use of a level of nuclear expression of TRF2 as a biomarker for determining a severity of an oral squamous cell carcinoma in an individual.

### LEGENDS TO THE FIGURES

FIG. 1 illustrates that TRF2 is a marker of poor prognosis independent of the tumor size marker. Univariate survival analysis investigating the impact of tumor size (T status, panel A), nodal status (N status, panel B) or TRF2 expression (panel C) on overall survival of patients with OSCC. “T1+T2” represents a cohort of individuals having small size tumors; “T3+T4” represents a cohort of individuals having large size tumors; “N0” represents a cohort of individuals having the tumor not spread to the lymph nodes; “N+” represents a cohort of individuals having the tumor spread to the lymph nodes; “0/+” represents a cohort of individuals having a TFR2 score of 0 and/or 1; “++/+++” represents a cohort of individuals having a TRF2 score of 2 and/or 3. Odds ratio for tumor size and TRF2 expression (panel D).

FIG. 2 illustrates that down-regulation of TRF2 expression or expression of a wild-type or a dominant negative form of TRF2 does not impair CAL33 proliferation. A. Human primary fibroblasts (FHN) (control; line 4), CAL33 cells expressing scramble shRNA (Addgene plasmid 1864; shC; line 1) or two independent shRNA sequences directed against TRF2 (sh1, sh2; lines 2 and 3, respectively) were tested for the presence of TRF2 by immunoblotting (upper panel). Actin is shown as a loading control (lower panel). B. Cumulative population doublings (CPD) of shC (diamonds), sh1 (triangles) and sh2 (squares) CAL33 cells. C. CAL33 cells stably transfected with a control expression vector (pWPIR-control; lines 1 and 2), a vector for TRF2 (pWPIR-TRF2; lines 3 and 4) or a dominant negative form of TRF2 (pWPIR-dnTRF2; lines 5 and 6) were tested for the presence of TRF2 by immunoblotting (upper panel). Actin is shown as a loading control (lower panel). D. Cumulative population doublings (CPD) of pWPIR-control (diamonds), pWPIR-TRFR2 (triangles) and pWPIR-dnTRF2 (squares) CAL33 cells.

FIG. 3 illustrates that down-regulation of TRF2 expression or expression of a wild-type or a dominant negative form of TRF2 does not impair CAL27 proliferation. A. Human primary fibroblasts (FHN) (control; line 1), CAL27 cells expressing scramble shRNA (Addgene plasmid 1864; shC; line 2) or two independent shRNA sequences directed against TRF2 (sh1, sh2; lines 3 and 4, respectively) were tested for the presence of TRF2 by immunoblotting (upper panel). Actin is shown as a loading control (lower panel). B. Cumulative population doublings (CPD) of shC (diamonds), sh1 (triangles) and sh2 (squares) CAL27 cells. C. CAL27 cells stably transfected with a control expression vector (pWPIR-control; lines 1 and 2), a vector for TRF2 (pWPIR-TRF2; lines 3 and 4) or a dominant negative form of TRF2 (pWPIR-dnTRF2; lines 5 and 6) were tested for the presence of TRF2 by immunoblotting (upper panel). Actin is shown as a loading control (lower panel). D. Cumulative population doublings (CPD) of pWPIR-control (diamonds), pWPIR-TRFR2 (triangles) and pWPIR-dnTRF2 (squares) CAL27 cells.

FIG. 4 illustrates that down-regulation of TRF2 modifies the secretome of CAL33. A. shC (left panel) and sh2 (right panel) CAL33 cell supernatants were tested for the presence of pro/anti-angiogenic, pro-inflammatory cytokines and growth factor using an antibody macroarray. Cytokines with a differential signal between shC and sh2 supernatants were squared (CXCL1 (1), IL6 (2), PDGF-BB (3) and RANTES (4)). B. Quantification of the signals shown in A; cytokines expressions from shC CAL 33 cells are represented by black bars, whereas cytokines expressions from sh2 CAL 33 cells are represented by white bars. The intensity of the signal obtained with shC CAL33 supernatants is shown as a reference (100%).

FIG. 5 illustrates that TRF2 down-regulation decreases tumor growth. 10⁶ CAL33LUC cells expressing shC (diamonds), sh1 (triangles) or sh2 (crosses) were subcutaneously injected into nude mice (n=10 per group). Bioluminescence was measured weekly as described previously (Grepin R et al. Oncogene 2012; 31(13):1683-94). Results are presented as the mean+SD. Statistical differences between the size of tumors of shC, sh1 and sh2 mice are presented: **P<0.01.

FIG. 6 illustrates that down-regulation of TRF2 does not modify the sensitivity to irradiation or to a treatment with 5 FU. Clonal growth of shC, sh1 and sh2 CAL33 after the indicated doses of irradiation (A, 4 and 8 grays) or in the absence (C, control; black bars) or presence (white bars) of 5 FU (B). The number of colonies in the absence of drugs for each cell line was considered as the reference value (100%). *P<0.05; ***P<0.001.

FIG. 7 illustrates that TRF2 down-regulation sensitizes CAL33 to suboptimal doses of erlotinib/Tarceva and cetuximab/Erbitux. Clonal growth of shC, sh1 and sh2 CAL33 cells in the absence (C; black bars) or presence of 6 nmol/L cetuximab (CX; gray bars) or of 0.1 μmol/L erlotinib (E; white bars). The number of colonies in the absence of drugs for each cell line was considered as the reference value (100%). ***P<0.001.

FIG. 8 illustrates that down-regulation of TRF2 sensitizes CAL33 cells to low doses of erlotinib. MTT tests were performed on shC and sh2 CAL33 cells in the absence (C; black bars) or presence of 0.1 μmol/L of erlotinib (E; gray bars). The mean OD after four days of untreated cells is used as the reference value (100%). **P<0.01.

FIG. 9 illustrates that down-regulation of TRF2 sensitizes CAL27 cells to cetuximab. Clonal growth of shC, and sh2 CAL27 in the absence (C; black bars) or presence of cetuximab (CX; gray bars). The number of colonies in the absence of drugs for each cell line was considered as the reference value (100%). *P<0.05.

### DETAILED DESCRIPTION OF THE INVENTION

Methods

Without wanting to be bound to a theory, the inventors provide herein an understanding of the association between TRF2 expression level in OSCC, aggressiveness of the tumor and treatment response in a retrospective cohort of patients and the biological mechanisms underlying this association. The experimental data in the examples section below have shown that over-expression of TRF2 is indicative of bad outcome in terms of survival independently of the size of the tumor. TRF2 abundance can be evaluated by immunohistochemistry and its detection would be informative in routine biopsies as a new biological parameter to make decision in treatment means (radiotherapy, chemotherapy and surgery) avoiding side effects for an increased comfort of patient and quality of life. It could constitute a component of individualized therapy. TRF2, namely Telomeric Repeat Factor 2, is a component of the shelterin complex, interacting with distal ends of chromosomes to maintain them folded in a conformation called “T-Loop”. When folded, the telomere is not recognized as DNA double strand damage by exonucleases and DNA repair systems.

TRF2 represents an essential link between telomeric DNA and other components of shelterin complex. In normal cells, TRF2 loss of function leads to activation of DNA repair systems specifically at telomeric loci, leading to cell cycle arrest, senescence or cell death. On the contrary, over-expression of TRF2 in the skin is associated with increased tumorigenesis (Munoz P et al. Nat Genet 2005; 37(10):1063-71).

Over-expression of TRF2 is observed in a large variety of human cancers, suggesting that TRF2 plays a key role in tumor initiation and development (Hu H et al. J Cancer Res Clin Oncol 2010; 136(9):1407-14; Da-Silva N et al. Dig Liver Dis 2010; 42(8):544-8; Dong W et al. Cancer Biol Ther 2009; 8(22):2166-74; Bellon M et al. Int J Cancer 2006; 119(9):2090-7; Oh B K et al. Am J Pathol 2005; 166(1):73-80; Nakanishi K et al. Clin Cancer Res 2003; 9(3):1105-11; Klapper W et al. Leukemia 2003; 17(10):2007-15; Matsutani N et al. Int J Oncol 2001; 19(3):507-12).

Inversely, Diehl et al. reported a lower expression of TRF2 within increased malignancy in breast cancer (Breast Cancer Res Treat. 2011; 127(3):623-30). These authors further suggested that higher levels of TRF2 protein may be important for protecting advanced cancer cells.

Within the more specific field of head and neck squamous cell carcinoma, apparent contradictory results have been observed with respect to the variation of TFR2 expression in cancer cells.

For example, Chuang et al. reported that TFR2 protein expression is decreased in oral cavity squamous cell carcinoma, and significantly associated with aggressive clinocopathological features (Exp and Ther Med. 2011; 2:63-67).

Oppositely, Padhi et al. recently reported that TRF2 is overexpressed by cancer cell in the head and neck squamous cell carcinoma (J Cancer. 2015; 6(2):192-202). Also, Sainger et al. reported that TRF2 is overexpressed by cancer cells from oral squamous cell carcinoma (OCCS), and that TRF2 could play a role in telomere length shortening (Biomarker Insights, 2007).

Nevertheless, none of these studies have established an association between TRF2 expression in tissue samples and clinical data such as cancer extension, overall survival and treatment response. Neither do these studies evaluate TRF2 as a prognostic marker or a predictive marker of sensitivity/resistance to targeted therapies.
Method for Predicting Likelihood to Efficiently Respond to an Anti-Cancer Treatment

The inventors have shown herein that the level of nuclear expression of TRF2 is correlated with the prognosis of outcome of cancer, in particular of oral squamous cell carcinoma (OSCC).

More precisely, the inventors have shown that the level of nuclear expression of TRF2 consists of a reliable prognosis marker of OSCC.

The present inventors have also shown that the level of nuclear expression of TRF2 consists of a reliable prognosis marker of the responsiveness of an individual affected with an OSCC to anti-cancer therapeutic treatments, such as therapeutic treatment based on antagonists of EGFR, notably anti-EGFR antibodies.

Furthermore, the inventors have shown that the level of nuclear expression of TRF2 consists of an independent prognosis marker of the outcome of OSCC or of responsiveness to an anti-OSCC therapeutic treatment.

As it is shown in the examples herein, the level of nuclear expression of TRF2 behaves as a reliable prognosis marker, irrespective of (i) the clinical stage of OSCC as determined by conventional clinical methods, (ii) irrespective of the level of migration of cancerous cells outside the tumor tissue (metastasis) and also (iii) irrespective of the tumour size.

Otherwise said, the level of nuclear expression of TRF2 consists of a novel and independent prognosis marker of OSCC. Moreover, the experimental data obtained by the inventors that the said marker is far more reliable that the previously available markers for OSCC.

In some embodiments, the level of nuclear expression of TRF2 may be used as the sole OSCC marker (i) in a method of prognosis of the outcome of an OSCC and (ii) in a method of prognosis of the responsiveness of a patient to an anti-OSCC therapeutic treatment.

In some embodiments, the level of nuclear expression of TRF2 may be used in combination with one or more already available OSCC markers in a method of prognosis of the outcome of an OSCC.

In a first aspect, the invention relates to an in vitro method for predicting a likelihood of an individual having a cancer to efficiently respond to an anti-cancer treatment, said method comprising the steps of:

a) measuring the level of nuclear expression of TRF2 in a sample obtained from said individual,

b) comparing the level obtained in step a) to a reference value, and

c) determining the predicted likelihood of said individual to efficiently respond to said anti-cancer treatment from the comparison performed in step b).

Within the scope of the invention, “an individual” is intended to mean any mammal, such as cat, dog, preferably a human being, irrespective of its age or gender. In particular, an individual encompassed by the invention is having an oral squamous cell carcinoma, and therefore the term “individual” also refers to an individual that may be undergoing or not an anti-cancer treatment, for example radiotherapy and/or chemotherapy.

Within the scope of the invention, the expression “likelihood to efficiently respond to” is meant to intend that the individual having a cancer may be subjected to stabilization, an alleviating, a curing or a reduction of the progression of the symptoms or the disease itself.

Within the scope of the invention, the term “cancer” encompasses, without restrictions, a colon cancer, a breast cancer, a bone cancer, an ovarian cancer, a skin cancer, a lung cancer, a kidney cancer, a lymphoma, a prostate cancer, a brain cancer, a bladder cancer, a liver cancer, a pancreatic cancer, an oral squamous cell carcinoma.

Within the scope of the present invention, the term “symptom” is intended to mean any noticeable response of the body to the cancer condition. In practice, a symptom encompasses a lesion, redness, a headache, dizziness, abdominal pain, coughing, vision troubles, swollenness, and the like.

In some embodiments, the cancer is an oral squamous cell carcinoma.

Within the scope of the present invention, the term “symptom”, when referring to an oral squamous cell carcinoma, is intended to mean any oral lesion that appears, within the mouth, i.e. between the vermilion border of the lips and the junction of the hard and soft palates or the posterior one third of the tongue, as area of erythroplakia or leukoplakia and may be exophytic or ulcerated.

Within the scope of the invention, the term “sample” is intended to mean any biological sample derived from an individual, such as a fluid, including blood, saliva; a tissue; a cell sample, an organ; a biopsy.

In certain embodiments, the sample comprises cancerous epithelial cells, preferably epithelial cancerous cells.

In certain embodiments, the sample is a tumor sample, preferably a tumor tissue biopsy or whole or part of a tumor surgical resection.

In some embodiments, a tumor sample, a tumor tissue biopsy or a tumor surgical resection may also comprise non-cancerous cells. In other words, the sample may be obtained by collecting both cancerous and non-cancerous, i.e. healthy, cells or tissue.

The sample may be collected according to conventional techniques in the art, and used directly for diagnosis or alternatively stored for subsequent diagnosis. A tumor sample may be fresh, frozen or paraffin-embedded. Usually, available tumor samples are frozen or paraffin-embedded, most of the time paraffin-embedded.

Within the scope of the invention, the expression “anti-cancer treatment” is intended to mean any medical treatment with a compound that specifically directly or indirectly targets the tumor in order to stabilize, alleviate, reduce the symptoms of the cancer or cure the cancer.

In some embodiments, individual having an oral squamous cell carcinoma that efficiently respond to an anti-cancer treatment may be characterized by a significant reduction of the size of the tumor, a reduction of the invasive properties of the tumor cells, a reduction of the migration properties of the tumor cells, a reduction of the mean cancerous cells counts, a variation of the expression of known biomarkers of the oral squamous cell carcinoma.

Each of these parameters may be evaluated accordingly to known methods routinely used in the art.

Within the scope of the invention, the expression “measuring the level of nuclear expression of TRF2” is intended to mean quantitatively or semi-quantitatively measuring either the level of TRF2 protein expression or the level of TRF2 gene expression in the nucleus of cells comprised within the sample.

If applicable, the nuclear fraction may be obtained from the sample comprising the cancerous cells to be assayed accordingly to the methods known in the art (Fujita et al. Nat Cell Biol. 2010 December; 12(12): 1205-1212; Wilkie and Schirmer. Chapter 2 Purification of Nuclei and Preparation of Nuclear Envelopes from Skeletal Muscle. The Nucleus: Volume 1: Nuclei and sub-nuclear components Hancock (ed.)).

According to a first embodiment of the invention, the expression “measuring the level of nuclear expression of TRF2” is intended to mean quantitatively or semi-quantitatively measuring the level of TRF2 protein nuclear expression.

In practice, the measurement of the level of TRF2 protein expression may be performed accordingly to any known method in the art.

In some embodiments, said method may comprise a step of contacting the sample with a compound capable of selectively binding to TRF2 protein, said compound being subsequently detected. For example, a compound capable of selectively binding to TRF2 protein may be an antibody, such as, for example, a monoclonal antibody or an aptamer.

In some embodiments, the level of TRF2 protein expression, notably of TRF2 protein nuclear expression, may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation; immunohistochemical staining. In some embodiments, a compound capable of selectively binding to TRF2 protein may be immobilized onto a suitable solid support, prior to its use.

In some preferred embodiment, the level of TRF2 protein expression, notably of TRF2 protein nuclear expression, may be measured by immunohistochemical staining, preferably by using a monoclonal antibody directed against TRF2 protein.

In some embodiments, monoclonal antibodies directed against TRF2 protein may be prepared accordingly to any method known from the state in the art, e.g. as described in Harlow and Lane (“Antibodies: A Laboratory Manual”, 2nd Ed; Cold Spring Harbor Laboratory Press, Cold Spring Harbor, N.Y., 1988).

In practice, in order to perform immunohistochemistry, tissue sections may be obtained from a individual and fixed onto a glass slide by any suitable fixing agent, such as e.g. alcohol, acetone, and paraformaldehyde, to which is reacted an antibody. Conventional methods for immunohistochemistry are described in Harlow and Lane (“Antibodies A Laboratory Manual”, Cold Spring Harbor Press, Cold Spring Harbor, N.Y., 1988), Ausbel et al. (“Current Protocols In Molecular Biology”, John Wiley and Sons, NY, 1987) or Brauer et al. (FASEB J, 15, 2689-2701; 2001).

In some embodiments, commercially available monoclonal antibody directed against TRF2 protein may be suitable, such as Clone 4A794.15 from Cayman Chemical®, clone 4A794 from Merck Millipore®, ab13579 from abcam®.

In some embodiments, the measure of the level of nuclear expression of TRF2 may be dependent of the intensity of the labelling obtained from the anti-TRF2 antibody and/or the number of cells expressing a noticeable amount of TRF2.

According to a second embodiment of the invention, the expression “measuring the level of nuclear expression of TRF2” is intended to mean quantitatively or semi-quantitatively measuring the level of TRF2 gene nuclear expression.

In practice, the measurement of the level of TRF2 gene expression may be performed accordingly to any known method in the art. It is understood by the man skilled in the art that the phrase “gene expression” relates to the synthesis of messenger RNA from the genes, in which a particular segment of DNA is copied into mRNA by the enzyme RNA polymerase. This mRNA synthesis called “transcription” is performed in the nucleus of the cell, and corresponds always a “nuclear expression”.

In some embodiments, the measurement of the level of TRF2 gene expression is performed by the measurement of the quantity of mRNA, notably by a method routinely used in the art. In practice, the total nucleic acids fraction contained in the sample may be obtained according to standard methods, such as using lytic enzymes or chemical solutions or extracted by nucleic-acid-binding resins following the manufacturer's instructions. The extracted mRNA may be subsequently detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous.

In some embodiments, other methods of amplification including ligase chain reaction (LCR), transcription-mediated amplification (TMA), strand displacement amplification (SDA) and nucleic acid sequence based amplification (NASBA) may be used.

In practice, probes for hybridization and/or amplification may be designed and obtained by any known method in the art.

In some other embodiments, the measurement of the level of TRF2 gene expression is performed by the DNA chip analysis technology (Hoheisel, Nature Reviews, Genetics, 2006, 7:200-210). Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling.

In some embodiments, measuring the level of nuclear expression of TRF2 in a sample obtained from said individual is performed in a sample comprising lymphocytes that have infiltrated the tumor.

Within the scope of the invention, the expression “reference value” is intended to mean a value representing a “standard” level of nuclear expression of TRF2, in conditions wherein the cells express TRF2 at a physiological level.

In some embodiments, said reference value may be measured in a sample obtained from one healthy individual, i.e. an individual without any medical condition or any history of medical condition.

In some embodiments, said reference value may represent a mean value measured in a plurality of samples obtained from one or more healthy individual(s). In some embodiments, a plurality of samples encompasses at least 2, at least 3, at least 5, at least 10, at least 15, at least 25, at least 50, at least 100, at least 250, at least 500 samples. In some embodiments, one or more healthy individual(s) encompasses at least 2, at least 3, at least 5, at least 10, at least 15, at least 25, at least 50, at least 100, at least 250, at least 500 healthy individuals.

In some embodiments, the mean reference value may be gathered from a plurality of samples stocked and periodically reevaluated.

In some embodiments, a reference value may be measured from the individual basal epithelial non-tumoral cells.

In some other embodiments, a reference value may be measured from the individual basal lymphocytes cells.

In some embodiments, a reference value is measured from a sample comprising cells line, such as e.g. lymphocytes and/or basal epithelial cells, and/or normal human fibroblasts.

In some embodiments, a reference value may be measured from a biopsy sample obtained from an individual having an oral squamous cell carcinoma, wherein said measure is performed in a portion of the biopsy that is free of cancerous cells.

In this preferred embodiments, both (i) the measured value on a portion of a biopsy sample comprising, or consisting essentially of, or alternatively consisting of, cancerous cells and (ii) the reference value measured on another portion of a biopsy sample comprising, or consisting essentially of, or alternatively consisting of, non-cancerous cells (e.g. lymphocyte cells), are obtained from the same individual having an oral squamous cell carcinoma. In some embodiments, the values (i) and (ii) may be measured on two distinct biopsy samples originating from the same patient, e.g. a non-cancerous biopsy sample and a cancerous biopsy sample, respectively. In some other embodiments, the values (i) and (ii) above may be measured on the same biopsy sample but (i) in a cancerous portion of the said biopsy sample and in a non-cancerous portion of the said biopsy sample, respectively.

In practice, both values (i) and (ii) may be normalized to one another.

Alternatively, in some other embodiments, said reference value may be measured in a sample obtained from a cohort of non-healthy individual, provided said sample(s) is/are free of any cancerous cells.

In some embodiments, a non-healthy individual encompasses an individual that does not have an oral squamous cell carcinoma.

In some embodiments, a non-healthy individual may be characterized by the presence of, without restriction, a cancer, such as a colon cancer, a breast cancer, an ovarian cancer, a skin cancer, a lung cancer, a kidney cancer, a lymphoma, a prostate cancer, a brain cancer, a bladder cancer, a liver cancer, a pancreatic cancer; an allergy, such as a air-borne allergy, a food allergy, a skin contact allergy; a respiratory insufficiency; a cardiac insufficiency; a renal insufficiency; a neurodegenerative disease; hypertension; bacterial or viral infection; Alzheimer disease; Parkinson disease; diabetes and the like.

In certain embodiments, said reference value may represent a mean value measured in a plurality of samples obtained from one or more non-healthy individual(s), provided said sample(s) is/are free of any cancerous cells, preferably free of any epithelial cancerous cells.

In some embodiments, a reference value may represent a mean value measured in a one or more sample(s) obtained from one or more healthy individual and one or more non-healthy individual(s), provided said sample(s) is/are free of any cancerous cells, preferably free of any epithelial cancerous cells.

In some embodiments, a reference value may also represent a value representing a “standard” level of nuclear expression of TRF2, from a sample obtained from an individual having an oral squamous cell carcinoma. Alternatively, a reference value may be also measured from oral squamous cell carcinoma commercial cell lines, such as, e.g. CRL-3212™ (2A3), CRL-3239™, CLR-3240™, HTB-43™ (FaDu), CRL-1628™ (SCC-25), CRL-1623™ (SCC-15), CRL-2095™ (CAL 27), CRL-1624™ (SCC-4), CRL-1629™ (SCC-9) and CCL-138™ (Detroit 562), from ATCC®; ACC 447™ (CAL 33) from The Leibniz Institute DSMZ (German Collection of Microorganisms and Cell Cultures GmbH).

In some preferred embodiments, the commercial cell line is selected in a group comprising CRL-2095™ (CAL 27), CCL-138™ (Detroit 562) and ACC 447™ (CAL 33).

In some embodiments, the level of TRF2 protein expression in one sample may be measured by immunohistochemical staining and further analysed by a semi-quantitative method comprising the scoring of said level of expression. In practice, nuclear stains related to TRF2 protein in one sample may be computed and analysed using a software or visually analysed and a score may be attributed to said sample. For example, a 4 grades scoring may be attributed accordingly to the measured level of TRF2 protein expression, namely:

0 absence of TRF2 nuclear expression;

+1, low TRF2 nuclear expression;

+2 mean TRF2 nuclear expression;

+3 strong TRF2 nuclear expression.

This method may be efficiently applied to both a sample of an individual having an oral squamous cell carcinoma, and a sample intended to provide a reference value as defined herein. Direct comparison between a score obtained from a sample of an individual having an oral squamous cell carcinoma, and a score obtained from a sample intended to provide a reference value is hence rendered facilitated.

In some embodiments, a reference value may be attributed a score of +2 (mean TRF2 nuclear expression).

In certain embodiments, the level of nuclear expression of TRF2 as measured in step a) lower than a reference value measured in a sample of a non-cancerous tissue is indicative of a good predicted likelihood of said individual having an oral squamous cell carcinoma to efficiently respond to said anti-cancer treatment.

Within the scope of the invention, the expression a “level of nuclear expression of TRF2 lower than a reference value” is intended to mean a level of nuclear expression of TRF2 that is at least 10% inferior as compared to the reference value. The expression “at least 10% inferior” encompasses at least 15% inferior, at least 20% inferior, at least 25% inferior, at least 30% inferior, at least 35% inferior, at least 40% inferior, at least 45% inferior, at least 50% inferior, at least 55% inferior, at least 60% inferior, at least 65% inferior, at least 70% inferior, at least 75% inferior, at least 80% inferior, at least 85% inferior, at least 90% inferior, at least 95% inferior, 100% inferior as compared to said reference value.

In other words, the measured level of nuclear expression of TRF2 in a sample of an individual having an oral squamous cell carcinoma may represent at most 90% of the reference value, which encompasses at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, at most 5%, 0% of the reference value.

Within the scope of the invention, the expression “sample of a non-cancerous tissue” is intended to mean any biological sample derived from an individual, such as a fluid, including blood, plasma, saliva, urine, seminal fluid; a tissue; a cell sample, an organ; a biopsy; provided that said sample does not comprise any cancerous cells.

In some embodiments, a level of nuclear expression of TRF2 as measured in a sample obtained from an individual having an oral squamous cell carcinoma lower than a reference value measured in a sample of a non-cancerous tissue may be achieved after the treatment of said individual with an inhibitor of TRF2 expression.

As shown in the examples section below, in vitro knock-down of TRF2 gene expression in CAL 33 line cells drastically increases the efficacy of erlotinib and cetuximab.

As mentioned above, a reference value may be attributed a score of +2 (mean TRF2 nuclear expression). Therefore, a score of 0 or +1, as the level of nuclear expression of TRF2 as measured in step a) is lower than the score of +2, as the reference value, and hence may be indicative of a good predicted likelihood of said individual having an oral squamous cell carcinoma to efficiently respond to an anti-cancer treatment.

In some embodiments, an individual having an oral squamous cell carcinoma responding efficiently to an anti-cancer treatment may be characterized by a better efficiency of the anti-cancer compound towards the disease.

Within the scope of the invention, “better efficiency of the anti-cancer compound towards the disease” is intended to mean that considering an identical dose of said anti-cancer compound, an individual having a lower level of nuclear expression of TRF2 as compared to a “standard” reference value, e.g. measured in a sample of a non-cancerous tissue, is likely to have the progression of the symptoms or the disease stop or decrease as compared to an individual having a level of nuclear expression of TRF2 similar, equal or higher as compared to a “standard” reference value.

In some embodiments, an individual having a lower level of nuclear expression of TRF2 as compared to a “standard” reference value, e.g. measured in a sample of a non-cancerous tissue may be characterized by requiring a lower dose of said anti-cancer compound to achieve similar results, as compared to the dose of said anti-cancer compound required to treat an individual having a level of nuclear expression of TRF2 similar, equal or higher as compared to a “standard” reference value.

In practice, a lower dose may encompass a measured efficient “IC50[lower TFR2]” that is significantly decreased as compared to a standard “IC50”, referenced for a given anti-EGFR inhibitor routinely used in cancer chemotherapy. In some embodiments, a lower dose encompasses a ratio IC50[lower TFR2]/standard IC50 ranging from 1:2 to 1:1000, which encompasses 1:3, 1:4, 1:5, 1:6, 1:7, 1:8, 1:9, 1:10, 1:15, 1:20, 1:25, 1:30, 1:35, 1:40, 1:45, 1:50, 1:75, 1:100, 1:125, 1:150, 1:175, 1:200, 1:250, 1:500 and 1:750.

In some embodiments, the anti-cancer treatment comprises the administration of an EGFR antagonist, which may be selected in a group comprising an EGFR tyrosine kinase inhibitor and a compound that specifically binds to EGFR.

Within the scope of the invention, the expression “EGFR antagonist” is intended to mean any compound that specifically directly or indirectly targets EGFR in order to inhibit its biological activity.

EGFR, or Epidermal Growth Factor Receptor, is a receptor of tyrosine kinase binding ligands of the EGF family that activates several signalling cascades. Known ligands of EGFR include EGF, TGFA/TGF-alpha, amphiregulin, epigen/EPGN, BTC/betacellulin, epiregulin/EREG and HBEGF/heparin-binding EGF.

Human EGFR (UNIPROT No P00533) accounts for at least 4 isoforms. Isoform 1 (UNIPROT No P00533-1) represents the canonical sequence of 1210 amino acids. The extracellular domain of human EGFR (isoform 1) may be represented by an amino acid sequence starting from the amino acid in position 25 to an amino acid in position 645. Isoform 2 (UNIPROT No PR00533-2) is a C-terminal truncated form of human EGFR of 405 amino acids and further characterized by a mutation of amino acids in position 404 and 405 from FL into LS. Isoform 3 (UNIPROT No PR00533-3) is a C-terminal truncated form of human EGFR of 705 amino acids. Isoform 4 (UNIPROT No PR00533-4) is a C-terminal truncated form of human EGFR of 628 amino acids.

Within the scope of the invention, EGFR belonging to other species or resulting from polymorphism are encompassed herein.

In some embodiments, individual having an oral squamous cell carcinoma that efficiently respond to an EGFR antagonist may be characterized by a significant reduction of the size of the tumor, a reduction of the invasive properties of the tumor cells, a reduction of the migration properties of the tumor cells, a reduction of the mean cancerous cells counts, a variation of the expression of known biomarkers of the oral squamous cell carcinoma.

In certain embodiments, the EGFR inhibitor is a tyrosine kinase inhibitor such as Erlotinib, Gefitinib, or Lapatinib.

In some embodiments, the EGFR inhibitor is a compound that specifically binds to EGFR.

In some embodiments, the EGFR inhibitor is an anti-EGFR antibody, most preferably a monoclonal antibody, such as Cetuximab, Panitumumab, Nimotuzumab (TheraCIM-h-R3), Matuzumab (EMD 72000) and Zalutumumab (HuMax-EGFr).

In some embodiments, the EGFR inhibitor is an aptamer specifically binding to EGFR, or to the extracellular domain of EGFR. Suitable aptamer may be obtained by any method routinely used in the art, for example le SELEX method, as originally described by Tuerk C and Gold L. (Science, 1990; 249(4968), 505-10), or an improved method thereof.

In some embodiments, an individual having a lower level of nuclear expression of TRF2 as compared to a reference value measured in a sample of a non-cancerous tissue is likely to efficiently respond to cetuximab and/or erlotinib.

In some embodiments, an individual having a lower level of nuclear expression of TRF2 as compared to a reference value measured in a sample of a non-cancerous tissue and likely to efficiently respond to cetuximab may be characterized by a ratio IC50[lower TFR2]/standard IC50 for cetuximab ranging from 1:2 to 1:10, which encompasses 1:3, 1:4, 1:5, 1:6, 1:7, 1:8 and 1:9.

In some embodiments, an individual having a lower level of nuclear expression of TRF2 as compared to a reference value measured in a sample of a non-cancerous tissue and likely to efficiently respond to erlotinib may be characterized by a ratio IC50[lower TFR2]/standard IC50 for erlotinib ranging from 1:2 to 1:100, which encompasses 1:3, 1:4, 1:5, 1:6, 1:7, 1:8, 1:9, 1:10, 1:15, 1:20, 1:25, 1:30, 1:35, 1:40, 1:45, 1:50 and 1:75.

As shown in the example section, reducing the level of expression of TRF2 in an individual having an oral squamous cell carcinoma may have an impact on the suboptimal doses of anti-cancerous agents that are of use for treating said individual.

In practice, the lower doses of anti-cancerous agent required to efficiently treat said individual having an oral squamous cell carcinoma and having lower level of expression of TRF2, as compared to an individual having an oral squamous cell carcinoma and having high level of expression of TRF2, may positively impact the side effects of said anti-cancerous agent.

In addition, lower level of TRF2 expression in an individual having an oral squamous cell carcinoma may further positively impact the likelihood of said individual to elicit an immune response against said anti-cancerous agent.

Therefore, lowering the level of expression of TRF2 in an individual having an oral squamous cell carcinoma may provide a benefit towards the sustainability of said anti-cancerous agent within the time course of the treatment.
Method for Predicting an Outcome

In another aspect, the invention relates to an in vitro method for predicting an outcome for an individual having an oral squamous cell carcinoma, said method comprising the steps of:

a) measuring a level of nuclear expression of TRF2 in a sample obtained from said individual,

b) comparing a level obtained in step (a) to a reference value, and

c) determining an outcome of said individual from said comparison performed in step b).

Within the scope of the invention, the term “outcome” may refer to an amelioration of the medical condition, i.e. the stabilization, an alleviating, a curing or a reduction of the progression of the symptoms or the disease itself. Such an outcome may be referred to a “good outcome” or “positive outcome”.

Within the scope of the invention, the term “outcome” may also refer to a worsening of the medical condition, i.e. an increase of the progression of the symptoms or the disease itself and even death of the individual. Such an outcome may be referred to a “bad outcome” or “negative outcome”.

In some embodiments, the outcome may be determined before a medical treatment intended to cure the oral squamous cell carcinoma. Said outcome may be determined within few days or few months prior initiating an anti-cancerous treatment intended to cure the oral squamous cell carcinoma.

In some embodiments, the outcome may be determined during the course of a medical treatment intended to cure the oral squamous cell carcinoma.

In some other embodiments, the outcome may be determined after a medical treatment intended to cure the oral squamous cell carcinoma. Said outcome may be determined within few days or few months after the end of an anti-cancerous treatment.

In some embodiments, any combination of determination of the outcome of an individual having an oral squamous cell carcinoma may be undertaken in order to adjust the medical treatment, which encompasses maintaining or stopping the treatment, increasing or decreasing the doses of the pharmaceutically active agent(s) to be administered, increasing or decreasing the time interval between two administrations of the pharmaceutically active agent(s).

In certain embodiments, the level of nuclear expression of TRF2 as measured in step a) lower than a reference value measured in a sample of a non-cancerous tissue is indicative of a good outcome for said individual.

Within the scope of the invention, the expression a “level of nuclear expression of TRF2 lower than a reference value” is intended to mean a level of nuclear expression of TRF2 that is at least 10% inferior as compared to the reference value. The expression “at least 10% inferior” encompasses at least 15% inferior, at least 20% inferior, at least 25% inferior, at least 30% inferior, at least 35% inferior, at least 40% inferior, at least 45% inferior, at least 50% inferior, at least 55% inferior, at least 60% inferior, at least 65% inferior, at least 70% inferior, at least 75% inferior, at least 80% inferior, at least 85% inferior, at least 90% inferior, at least 95% inferior, 100% inferior as compared to said reference value.

Within the scope of the invention, the expression “good outcome” encompasses any amelioration of the medical condition, including stabilization, an alleviating, reduction of the progression of the symptoms or the disease itself or a total remission.

In some embodiments, a good outcome may be evaluated accordingly to the RECIST criteria (Eisenhauer et al, European Journal of Cancer, 2009, 451228-247).

In solid tumors, the RECIST criteria provide an international standard based on the presence of at least one measurable lesion. “Complete response” means disappearance of all target lesions (total remission); “partial response” means 30% decrease in the sum of the longest diameter of target lesions, “progressive disease” means 20% increase in the sum of the longest diameter of target lesions, “stable disease” means changes that do not meet above criteria.

Within the scope of the invention, the expression “good outcome” also encompasses a benefit in term of overall survival prognosis. In some embodiments, an overall survival prognosis benefit may account for at least 6 months of gain of life expectancy without aggravation of the symptoms of the disease, which includes, at least 9 months, 12 months, 15 months, 18 months, 24 months, 36 months, 48 months, 60 months, 5 years, 6 years, 7 years, 8 years of gain of life expectancy.

In some embodiments, a good outcome may account for an overall survival of at least 50% at 9 months, 12 months, 15 months, 18 months, 24 months, 36 months, 48 months, 60 months, 5 years, 6 years, 7 years, 8 years.

In another aspect, the invention relates to a method for treating an individual having an oral squamous cell carcinoma comprising the administration of an inhibitor of the TRF2 gene expression.

In some embodiments, the method also comprises the administration of an anti-cancerous compound.
Method for Screening Anti-Cancer Compound for Ameliorating the Outcome of an Individual Having an Oral Squamous Cell Carcinoma

In another aspect, the invention relates to an in vitro method for screening an anti-cancer compound for treating and/or ameliorating the outcome of an individual having an oral squamous cell carcinoma, said method comprising the steps of:
a) contacting a TRF2 knock down cell line of oral squamous cell carcinoma with a candidate anti-cancer compound;
b) measuring a parameter linked with the;
c) comparing the parameter from step c) with the same parameter measured in the absence of said candidate anti-cancer compound.

In some embodiments, a cell line of oral squamous cell carcinoma may be obtained after a biopsy or alternatively from a commercial provider. The cell line may be cultivated under standard conditions described in the state of the art.

In some embodiments, the cell line is selected from oral squamous cell carcinoma commercial cell lines, such as, e.g. CRL-3212™ (2A3), CRL-3239™, CLR-3240™, HTB-43™ (FaDu), CRL-1628™ (SCC-25), CRL-1623™ (SCC-15), CRL-2095™ (CAL 27), CRL-1624™ (SCC-4), CRL-1629™ (SCC-9) and CCL138™ (Detroit 562), from ATCC®; ACC 447™ (CAL 33) from The Leibniz Institute DSMZ (German Collection of Microorganisms and Cell Cultures GmbH).

In some preferred embodiments, the commercial cell line is selected in a group comprising CRL-2095™ (CAL 27), CCL-138™ (Detroit 562) and ACC 447™ (CAL 33).

In practice, the TFR2 knock down of the cell line may be obtained by any methods in the art, such as gene knock out, or silencing, e.g. by the use of any suitable inhibitor of the TRF2 gene expression, preferably selected in a group comprising an antisense DNA, an antisense RNA, a double stranded RNA, a mi RNA, a siRNA, a shRNA, an aptamer specifically binding to the mRNA encoded by a TRF2 gene and a non-specific inhibitor of TRF2 gene expression.

In some embodiments, TRF2 knock down may be performed by the well documented CRISPR-Cas9 technology (WO 2013/176772; U.S. Pat. No. 8,697,359; WO 2014/089290).

In some other embodiments, TRF2 knock down may be performed by the use of nucleases technologies, such as e.g. the zinc finger technology or the Transcription Activator-Like Effector Nuclease (TALEN) technology Ul Ain et al. J Control Release. 2015 May 10; 205:120-7; Boettcher and McManus. Mol Cell. 2015 May 21; 58(4):575-585. Wright et al. Biochem J. 2014 Aug. 15; 462(1):15-24).

Within the scope of the invention, a “parameter linked with the oral squamous cell carcinoma” is intended to mean any measurable parameter that is correlated with the occurrence of the disease itself.

In some embodiments, said parameter may be, without any limitation, the mean size of the cells, the mean morphology of the cells, the invasion properties of the cells, the proliferative properties of the cells, the migration properties of the cells, the variation of expression of a specific biomarker, the secretion of a specific biomarker and the like.

In some embodiments, any observed variation between the measured parameter in the presence and in the absence of the candidate anti-cancer compound may be indicative of the efficacy of said anti-cancer compound for its use in the treatment of an oral squamous cell carcinoma in an individual in need thereof.

In a still another aspect, the invention relates to an in vivo method for screening an anti-cancer compound for treating and/or ameliorating the outcome of an individual having an oral squamous cell carcinoma, said method comprising the steps of:
a) contacting a TRF2 knock down non-human animal model for oral squamous cell carcinoma with a candidate anti-cancer compound;
b) measuring a parameter linked with the;
c) comparing the parameter from step c) with the same parameter measured in the absence of said candidate anti-cancer compound.

In some embodiments, the TRF2 knock down non-human animal model for oral squamous cell carcinoma may be obtained accordingly to standard techniques for providing transgenic non-human model.

In some other embodiments, the non-human animal model may be injected with oral squamous cell carcinoma cells and further treated intra-tumorally or systemically with an inhibitor of TRF2 gene expression.

In some embodiments, the non-human animal model is a rodent, such as a mouse, a rat.
Uses

In a one aspect, the invention relates to an inhibitor of the TRF2 gene expression for use in the treatment of an individual having an oral squamous cell carcinoma.

In some embodiments, the dosage regimen of said inhibitor to be administered to an individual in need thereof depends of the age, the gender, the weight and the further medical condition or the medical history of said individual and may be ranging from 10 μg to 1000 mg, which encompasses 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg.

In some embodiments, the frequency of administration said inhibitor to be administered to an individual in need thereof depends of the age, the gender, the weight and the further medical condition or the medical history of said individual and may be ranging from once, twice, three times a day; once, twice, three times a week.

In some embodiments, the dosage regimen and/or the frequency of administration said inhibitor to be administered to an individual in need thereof may be adjusted at any time depending on the progression of the symptoms within the time course of the treatment.

In one embodiment, the individual that is treated with an inhibitor of the TRF2 gene expression has been previously identified as an individual having a good likelihood to efficiently respond to an anti-cancer treatment, according to the previously disclosed method.

In another embodiment, the individual that is treated with an inhibitor of the TRF2 gene expression has been previously identified as an individual having a poor likelihood to efficiently respond to an anti-cancer treatment, according to the previously disclosed method. Within the scope of the invention, the term “inhibitor of TRF2” encompasses specific inhibitors of TRF2 gene expression such as an antisense DNA, an antisense RNA, a double stranded RNA, a mi RNA, a siRNA, a shRNA, and an aptamer, wherein said inhibitor specifically binds to the mRNA encoded by a TRF2 gene.

In some embodiments, said inhibitor of the TRF2 gene expression is selected from an antisense DNA, an antisense RNA, a double stranded RNA, a mi RNA, a siRNA, a shRNA, an aptamer specifically binding to the mRNA encoded by a TRF2 gene and a non-specific inhibitor of TRF2 gene expression.

In some embodiments, a mi RNA (micro RNA), a siRNA (small interfering RNA), a shRNA (short hairpin RNA), an aptamer may be designed accordingly to the methods known in the art, as to specifically bind to a mRNA encoded by a TFR2 gene in order to provide an in vivo TRF2 gene inhibition or TRF2 silencing.

In some embodiments, a non-specific inhibitor of TRF2 gene expression may act on any gene or protein involved in the regulation of TRF2 expression.

In some preferred embodiments, said inhibitor of the TRF2 gene expression is a shRNA, in particular a shRNA comprising the sequence SEQ ID No2 or SEQ ID No3.

In another aspect, the invention relates to an ex vivo or in vitro use of a level of nuclear expression of TRF2 as a biomarker for predicting an outcome for an individual having an oral squamous cell carcinoma.

In a one aspect, the invention relates to an ex vivo or in vitro use of a level of nuclear expression of TRF2 as a biomarker for predicting a likelihood of an individual having an oral squamous cell carcinoma to efficiently respond to an anticancer treatment.

Within the scope of the invention, the term “biomarker” is intended to mean any measurable parameter that is significantly correlated with a physiological or pathological event in a living body.

As shown in the examples section below, the level of nuclear expression of TRF2 is significantly correlated with the efficacy of anti-cancer treatment, namely a treatment comprising the administration of an EGFR antagonist, to eradicate cancer cells.

In some embodiments, the level of nuclear expression of TRF2 for use as a biomarker may be combined to one or more other known biomarker(s) for predicting an outcome for an individual having an oral squamous cell carcinoma or for predicting a likelihood of an individual having an oral squamous cell carcinoma to efficiently respond to an anti-cancer treatment.

In some embodiments, other biomarkers may be selected in a group comprising the size of the tumor, the nodal status, the invasion properties, the level of expression of RANTES, the level of expression of IL6, the level of expression of IL8 the level of expression of CXCL9, the level of expression of CXCL10, the level of expression of PDGFBB, the level of expression of VEGF and the level of expression of GRO-alpha.

Within the scope of the invention, the “nodal status” relates to the clinical observation of a presence or an absence of lymph node in an individual having a cancer. In practice, if the cancer has spread into the lymph nodes, the cancer is termed “node-positive”, whereas the cancer is termed “node-negative” if it has not spread into the lymph nodes.

In a one aspect, the invention relates to an ex vivo or in vitro use of a level of nuclear expression of TRF2 as a biomarker for determining a severity of an oral squamous cell carcinoma in an individual.

As shown in the examples section, the level of nuclear expression of TRF2 is significantly correlated with the survival of the individuals having an oral squamous cell carcinoma. Therefore, the level of nuclear expression of TRF2 is indicative of the severity of the oral squamous cell carcinoma.

It is well documented in the art that an oral squamous cell carcinoma may be subcategorized in 4 stages, depending of the progression of the cancer within the individual.

A stage I cancer may be defined as presenting a tumor that is less than 2 centimetres in size, and has not spread to lymph nodes in the area.

A stage II cancer may present tumors that are more than 2 centimetres in size, but less than 4 centimetres, and has not sill spread to lymph nodes in the area.

A stage III cancer may be characterized by either (i) a tumor being more than 4 centimeters in size, or (ii) a tumor being of any size but has spread to only one lymph node on the same side of the neck as the cancer, or (iii) a lymph node that contains cancer measuring no more than 3 centimetres.

A stage IV cancer may be defined by either (i) the tumor having spread to tissues around the lip and oral cavity, or (ii) the lymph nodes in the area may or may not contain a tumor, or (iii) the tumor being of any size and having spread to more than one lymph node on the same side of the neck as the tumor, to lymph nodes on one or both sides of the neck, or to any lymph node that measures more than 6 centimetres, or (iv) the tumor having spread to other parts of the body.

In some embodiments, a measured level of nuclear expression of TRF2 in an individual having an oral squamous cell carcinoma comprised from 1.5 to 3 times as compared to a “standard” reference value, e.g. measured in a sample of a non-cancerous tissue may be indicative of a stage I cancer.

Within the scope of the invention the expression “from 1.5 to 3 times” encompasses 1.6, 1.7, 1.8, 1.9, 2.0, 2.1, 2.2, 2.3, 2.4, 2.5, 2.6, 2.7, 2.8 and 2.9 times.

In some embodiments, a measured level of nuclear expression of TRF2 in an individual having an oral squamous cell carcinoma comprised from 3.1 to 5 times as compared to a “standard” reference value, e.g. measured in a sample of a non-cancerous tissue may be indicative of a stage II cancer. Within the scope of the invention the expression “from 3.1 to 5 times” encompasses 3.2, 3.3, 3.4, 3.5, 3.6, 3.7, 3.8, 3.9, 4.0, 4.1, 4.2, 4.3, 4.4, 4.5, 4.6, 4.7, 4.8 and 4.9 times.

In some embodiments, a measured level of nuclear expression of TRF2 in an individual having an oral squamous cell carcinoma comprised from 5.1 to 7 times as compared to a “standard” reference value, e.g. measured in a sample of a non-cancerous tissue may be indicative of a stage III cancer.

Within the scope of the invention the expression “from 3.1 to 5 times” encompasses 5.2, 5.3, 5.4, 5.5, 5.6, 5.7, 5.8, 5.9, 6.0, 6.1, 6.2, 6.3, 6.4, 6.5, 6.6, 6.7, 6.8 and 6.9 times.

In some embodiments, a measured level of nuclear expression of TRF2 in an individual having an oral squamous cell carcinoma at least 7.1 times as compared to a “standard” reference value, e.g. measured in a sample of a non-cancerous tissue may be indicative of a stage IV cancer.

Within the scope of the invention the expression “at least 7.1” encompasses at least 7.5, at least 8, at least 9, at least 10, at least 25, at least 50, at least 100.
Kits

In another aspect, the invention relates to a kit for use in the treatment of an individual having an oral squamous cell carcinoma comprising:

an inhibitor of the TRF2 gene expression in a physiologically acceptable excipient, and,

an anti-cancer compound in a physiologically acceptable excipient.

In one embodiment, the individual that is treated with an inhibitor of the TRF2 gene expression has been previously identified as an individual having a good likelihood to efficiently respond to an anti-cancer treatment, according to the previously disclosed method.

In another embodiment, the individual that is treated with an inhibitor of the TRF2 gene expression has been previously identified as an individual having a poor likelihood to efficiently respond to an anti-cancer treatment, according to the previously disclosed method.

In some embodiments, the inhibitor of the TRF2 gene expression and/or the anti-cancerous compound may be further formulated in a composition comprising at least one physiologically acceptable excipient.

Suitable physiologically acceptable excipients include, but are not limited to, any and all conventional solvents, dispersion media, fillers, solid carriers, aqueous solutions, coatings, antibacterial and antifungal agents, isotonic and absorption delaying agents, and the like. Suitable physiologically acceptable excipients include, for example, water, saline buffer, phosphate buffered saline buffer, dextrose, glycerol, ethanol and the like, as well as combinations thereof. Physiologically acceptable excipients may further comprise minor amounts of auxiliary substances such as wetting or emulsifying agents, preservatives, which may enhance the shelf life or effectiveness of the inhibitor of the TRF2 gene expression and/or the anti-cancerous compound. The preparation and use of physiologically acceptable excipients is well known in the art.

Such inhibitor of the TRF2 gene expression and/or such anti-cancerous compound may be administered simultaneously or in a delayed manner.

Administration of said inhibitor of the TRF2 gene expression and/or said anti-cancerous compound may rely upon a parenterally, e.g., by injection, either subcutaneously or intramuscularly, as well as orally, intranasally or intratumoral administration. Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium carbonate, and the like, without limitation.

An inhibitor of the TRF2 gene expression has been previously defined in the present specification.

In certain embodiments, said inhibitor of the TRF2 gene expression is selected from an antisense DNA, an antisense RNA, a double stranded RNA, a mi RNA, a siRNA, a shRNA, an aptamer specifically binding to the mRNA encoded by a TRF2 gene and a non-specific inhibitor of TRF2 gene expression.

In certain embodiments, said anti-cancer compound is an EGFR antagonist.

In certain embodiments, said EGFR antagonist is selected in a group comprising an EGFR tyrosine kinase inhibitor and a compound that specifically binds to EGFR, preferably to the extracellular domain of EGFR.

In certain embodiments, said EGFR antagonist is selected in a group comprising Erlotinib, Gefitinib, Lapatinib, Cetuximab, Panitumumab, Nimotuzumab, Matuzumab and Zalutumumab.

In another aspect, the invention relates to a kit for predicting a likelihood of an individual having an oral squamous cell carcinoma to efficiently respond to an anti-cancer treatment, said kit comprising:

a) reagents for measuring the level of nuclear expression of TRF2 in a sample obtained from said individual,

b) reagents for determining at least one other parameter positively or negatively correlated to response to said anti-cancer treatment.

Within the scope of the invention, the term “reagent” may encompass a nucleic probe, a fluorescent probe, an antibody, an aptamer, a liquid solution, and the like.

In some embodiments, the kit may further comprise a basal epithelial cells line, as for measuring a reference value for nuclear expression of TRF2.

### EXAMPLES

1—Materials and Methods

1.1—Patients and Tissue Samples

62 OSCC tissue samples were obtained from patients who had been diagnosed at Centre Antoine Lacassagne and Hospital St Roch between 1996 and 2011. All patients were confirmed by histology and gave their consent. Tumor sections of OSCC patients were previously examined to be sufficient for evaluation by immunohistochemistry at Center Antoine Lacassagne and Laboratory of histology of Hospital Pasteur. Clinical data such as TNM classification, diagnosis, date of diagnosis, treatment and last known status of the patient were obtained by searching through the clinicom® database. Survival terms were calculated from the date of diagnosis of oral carcinoma. Key patients' characteristics in this study are summarized in Table 1.

TABLE 1
Clinical characteristics of the patients.

| Variable | Quantity | Frequency (%) |
|---|---|---|
| Sex | | |
| Male | 44 | 71 |
| Female | 18 | 29 |
| T stage | | |
| unknown | 10 | 16 |
| 1 | 12 | 19 |
| 2 | 8 | 13 |
| 3 | 9 | 15 |
| 4 | 23 | 37 |
| N stage | | |
| unknown | 13 | 21 |
| 0 | 31 | 50 |
| 1 | 2 | 3 |
| 1c | 1 | 2 |
| 2 | 3 | 5 |
| 2b | 7 | 11 |
| 2c | 3 | 5 |
| 3 | 2 | 3 |
| M stage | | |
| unknown | 21 | 34 |
| 0 | 41 | 66 |
| Differentiation | | |
| unknown | 30 | 48 |
| high grade dysplasia | 1 | 2 |
| 1 | 5 | 8 |
| 2 | 5 | 8 |
| 3 | 21 | 34 |
| Keratinization | | |
| unknown | 32 | 52 |
| no | 10 | 16 |
| yes | 20 | 32 |
| Inflammation | | |
| unknown | 39 | 63 |
| 0 | 11 | 18 |
| 1 | 4 | 6 |
| 2 | 8 | 13 |
| Surgery | | |
| unknown | 4 | 6 |
| surgery | 39 | 63 |
| no surgery | 19 | 31 |
| Radiotherapy | | |
| unknown | 5 | 8 |
| radiotherapy | 38 | 61 |
| no radiotherapy | 19 | 31 |
| Chemotherapy | | |
| unknown | 5 | 8 |
| chemotherapy | 14 | 23 |
| no chemotherapy | 43 | 69 |

The mean age of the OSCC patients was 60.5 years. Most patients were men (71%) with a sex ratio of 2.45. Most of the tumors were invasive (44%) (T4 stage) and measured more than 4 cm. 63% of the patients didn't present any lymphatic node invasion. Most of the patients were treated by surgery (67%) and radiotherapy (67%) but chemotherapy was used in only 14 patients (25%). The median survival time (MST) was 45.3 months with the 1 year survival rate being 78.5%.

1.2—Cell Lines

Two human head and neck cancer cell lines from ATCC® were studied:

CAL 33™, a human OSCC of the tongue mutated for p53, established in Center Antoine Lacassagne, Nice, France (Gioanni Jet al. Eur J Cancer Clin Oncol 1988; 24(9):1445-55).

CAL 27™, a human OSCC, established in Center Antoine Lacassagne, Nice, France.

To further analyze the effect of TRF2 knock-down on cell proliferation, invasion capabilities and treatment response, CAL 33 cells expressing shRNA against TRF2 and dominant negative forms of TRF2 were generated (Biroccio A et al. Nat Cell Biol 2013; 15(7):818-28). CAL 33 cells were infected using lentiviral vector (pLKOpuro-3×LacO, Sigma). The plasmids contained scramble (shC; SEQ ID No1) or TRF2-specific shRNA, sh1 (SEQ ID No2) or sh2 (SEQ ID No3).

Nucleic acids sequences used therein are summarized in
the Table 2 below:

| SEQ | | |
|---|---|---|
| ID No | Sequence | Observation |
| 1 | CCTAAGGTTAAGTCGCCCTCGCTCGAGCGAGGGCG | DNA encoding shC; |
| | ACTTAACCTTAGG | scramble shRNA |
| | | (Addgene plasmid |
| | | 1864) |
| 2 | GCCAGAATATCATTAGCGTTTCTCGAGAAACGCTA | DNA encoding sh1; |
| | ATGATATTCTGGC | TRF2-specific |
| | | shRNA |
| 3 | GCGCATGACAATAAGCAGATTCTCGAGAATCTGCT | DNA encoding sh2; |
| | TATTGTCATGCGC | TRF2-specific |
| | | shRNA |

1.3—In Vitro Proliferation Assays

For cumulative population doublings assays (Nekanti U et al. Int J Biol Sci 2010; 6(5):499-512.) 10⁵ cells were grown in 7.5% FCS-DMEM medium for 28 days at 37° C. and 5% CO₂ in Petri boxes 100 mm diameter. Cells were counted each day (Coulter, Beckman) and media were changed every five days. Cells at each passage was calculated as a ratio of total number of cells harvested to total number of cells seeded multiplied by the total number of cells from the previous passage. Population doublings were calculated using the formula:

X=[log₁₀(NH)−log₁₀(NI)]/log₁₀(2),

in which NI is the inoculum cell number and NH the cell harvest number.

To yield the cumulated doubling level, the population doubling for each passage was calculated and then added to the population doubling levels of the previous passages. The population doubling time was obtained by the formula:

TD=tplg2/(lgNH−lgNI),

In which NI is the inoculum cell number; NH is the cell harvest number and t is the time of the culture (in hours).

The mean and standard deviation were calculated for three independent experiments. Statistical analysis was carried out using a t test. For clonogenicity assays cells were, cells (2×10³) were seeded onto 60 mm dishes. Twenty-four hours later after cell attachment, medium was replaced with regular DMEM supplemented with 10% serum, for 10 d of growth in the presence or in the absence of erlotinib/Tarce (1 μmol/L). Dishes were then stained with Giemsa (Fluka) and plates were scanned in order to analyze the results by computer with ImageJ software (NIH, USA). The concentration of erlotinib/Tarceva which reduces tumor cell growth, was assessed by using the 3-[4,5-dimethylthiazol-2yl]-diphenyltetrazolium bromide (MTT) colorimetric assay (Sigma, Lyon, France) according to the manufacturer's instructions.

1.4—In Vitro Cell Migration and Invasion

To obtain spheroïds, 4000 cells were seeded in 20 μL hanging drops of DMEM supplemented with 7.5% fetal bovine serum (DMEM-7.5% FCS). After 7 days, spheroïds were transferred into matrigel (Corning Inc.) diluted in DMEM-3% FCS and were cultured for 15 days. Pictures were taken with an AMG Evos microscope 40× objective (Thermo Fisher Scientific Inc) and the diameter of spheroids was measured using ImageJ software (NIH, USA).

1.5—Western Blots

The following antibodies were used for Western blotting: anti-phospho ERK 1,2 (Sigma St Louis, Mo.), anti-phospho Akt, anti-Akt, anti-EGFR (Cell Signaling, Cambridge, UK,), anti-ERKs (Santa Cruz Biotechnology, Santa Cruz, Calif.)), anti-TRF2 (Novus bio, Cambridge, UK) and alpha-tubulin (Fischer scientific, Illkirch France).

1.6—Tumor Xenograft Formation and Size Evaluation

10⁶ cells were injected subcutaneously into the flanks of 5-week-old nude (nu/nu) female mice (Janvier). Bioluminescence was quantified using the In Vivo Imaging System (Perkin Elmer) according to the manufacturer's instructions. Tumor volume [(v ¼ L l² 0.52] was determined in parallel using a caliper. There was a linear relationship between values for bioluminescence and the tumor volume.

1.7—Immuno-Histochemical Staining of TRF2 in Human and Mouse Tissue Sections

Immuno-histochemical staining for TRF2 was carried out in 3 μm tissue sections from formalin-fixed, paraffin-embedded tissue blocks. Histopathological analysis was conducted in 3 μm tissue sections colored with Masson's trichrome (blue collagen) and scanned with Leica Slide Path. For each tumor, pictures were taken and the following parameters were analyzed using Leica Slide Path Gateway software: total area of the section, area of necrosis, presence of white blood cells inflammatory infiltrate, presence of red blood cells extravasation, thickness of collagen around vessels, number of vessels.

Endogenous peroxidase inactivation in PBS for 30 minutes of deparaffinized sections was carried out followed by re-hydration (Dako 48 link autostainer, Dako, Capinterie, Calif., USA) and heat unmasking of antigens for 20 minutes at 97° C. in a pH=9 buffer solution (PT link Dako device). Incubation was carried out with monoclonal antibody anti-TRF2 diluted at 1:100 for 20 minutes at room temperature. After application of the secondary antibody, the tissues sections were then treated with 3′,3′-diaminobenzidine chromogen and counterstained for nucleus with Mayer's hematoxylin. TRF2 reactivity on lymphocytes and/or basal epithelial cells was considered as internal positive control. Nuclear expression of TRF2 was semi-quantitatively analyzed and verified independently by two pathologists (D. Ambrosetti and A. Sudaka) and two surgeons (H. Raybaud and Y. Benhamou). In cases of differences a third person was consulted to read the sections.

Tumors were classified as follows:

0 absence of nuclear expression in tumor cells;

+1, low expression;

+2 mean expression;

+3 strong expression.

When tissue sections contained 2 different scores, the upper score was chosen if more of 30% of stained nuclei were concerned.

1.8—Immunofluorescence

Tumor sections were handled as described previously for immunofluorescence experiments (Perri F et al. Anticancer Agents Med Chem 2013; 13(6):834-43.). Sections were incubated with rat monoclonal anti-mouse CD31 (clone MEC 13.3; BD Pharmingen) and monoclonal anti-alpha-smooth muscle actin Sigma (A2547, 1:1000; Sigma, France).

1.9—Statistical Analysis Method

The end point for all analyses was overall survival (OS) which was defined as time from primary diagnosis to the death of the patient. For patients who did not deceased, the time from primary diagnosis to the last documented follow-up was used. The OS rates were calculated according to the Kaplan Meier method. The hazard ratio (HR) between different groups defined by TRF2 score (with corresponding confidence intervals) was determined by the cox regression model. The categorization of the immunohistochemistry factors in subgroups was predefined independently of results of analyses. All analyses are based on 62 patients for whom all immuno-histochemical determinations and clinical histories are completely documented. For univariate and multivariate analysis, the 0 and 1+ and the 2+ and 3+ tumor's scores were combined into independent groups representing “TRF2 negative” and “TRF2 positive”.

1.10—Evaluation of DNA Damage by Immunofluorescence.

Slides were fixed with methanol at −20° C. or 4% formaldehyde at room temperature for 15 min, and then incubated for 1 h with blocking buffer (0.8× PBS, 50 mM NaCl, 0.5% Triton X-100 and 3% milk), followed by incubation overnight at 4° C. with −53BP1 (NB100-305; Novus Biologicals) antibodies. The cells were then washed with 0.8× PBS, 50 mM NaCl and 0.1% Triton X-100 and incubated with anti-mouse Alexa488 (A21202; Life Technologies) and anti-rabbit Alexa555 (A-31572; Life Technologies) antibodies. After washing with 0.8× PBS, 50 mM NaCl, and 0.1% Triton X-100, the nucleus was labeled with DAPI.

1.11—Determination of CAL33 Secretome by Macroarray

10⁶ shC and sh2 CAL33 cells were grown for 48 h in normal conditions. Cell supernatants were centrifuged and incubated on a specific membrane for testing cytokine expression as indicated by the manufacturer protocol (Angiogenesis array I, Tebu-bio). The membrane was revealed by chemoluminescence. Quantification of the intensity of the different spots was performed using an Odyssey imager (LI-COR biotechnology). The relative intensity was normalized to negative and positive controls included on the membrane.

2—Results

2.1—TRF2 Expression and Survival of OSCC Patients

No previous scoring of TRF2 expression in OSCC by immuno-histochemistry was available. Therefore a score inspired by the HER2 evaluation in breast cancers was established to determine for TRF2. The prognostic significance of TRF2 expression level and other pathologic factors in patients with OSCC was evaluated by univariate analysis.

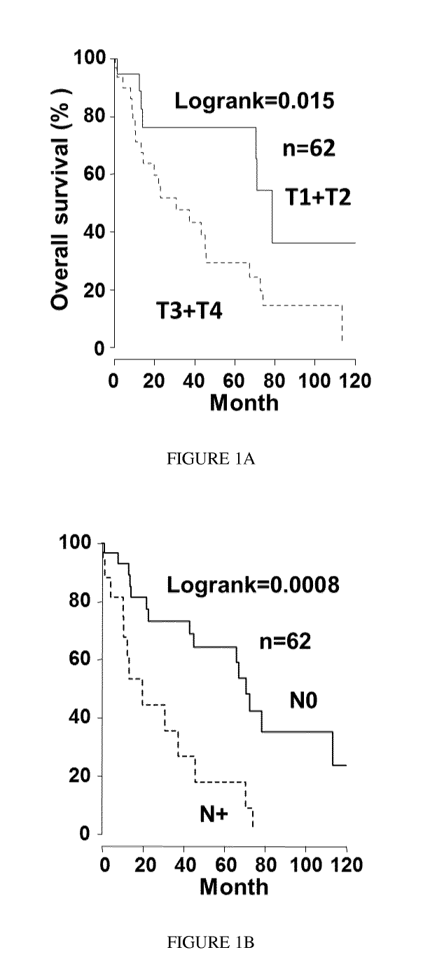

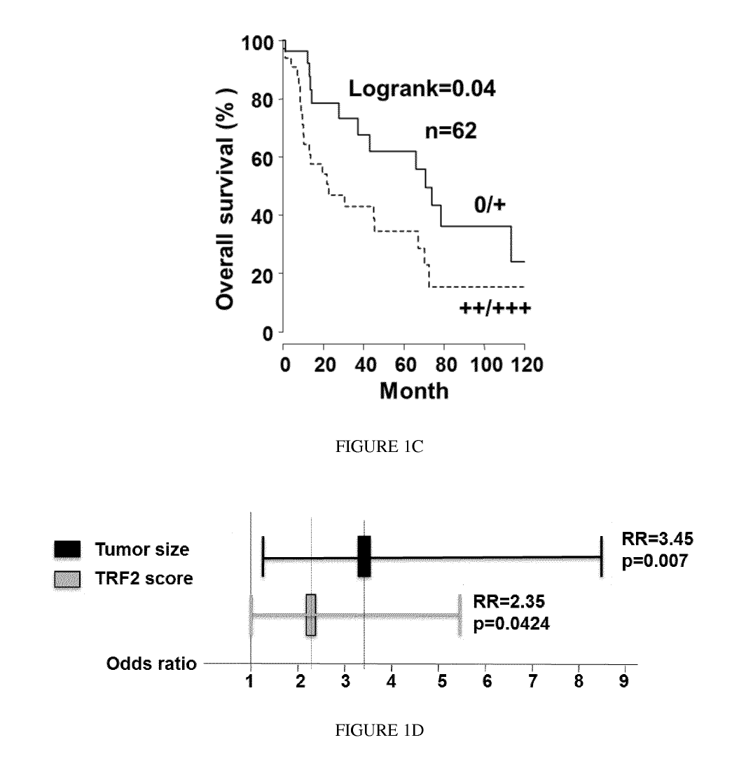

The immuno-histochemistry data show that tumor size (T) and nodal status (N), both known as poor prognostic factors for patient's outcome, were also significantly correlated to overall survival (p=0.015 and 0.0008, respectively) (FIGS. 1A and 1B). 34 patients were TRF2 positive and 24 patients were TRF2 negative according to the above defined scoring method. A significant relationship between TRF2 nuclear expression in OSCC tissue sections of patients and survival was observed (median survival time 71 months for 0-1+ patients versus 24 months for 2+-3+ patients p=0.0418), introducing a new biological prognostic marker for OSCC (FIG. 1C). Multivariate analysis showed that TRF2 score (OR=2.35 [1.01-5.45] 95% CI, p=0.0424) was independent of tumor size (OR=3.45 [1.387-8.628] 95% CI, p=0.007) (FIG. 1D).

2.2—In Vitro Effects of Modulation of TRF2 Expression/Activity in OSCC Cell Lines

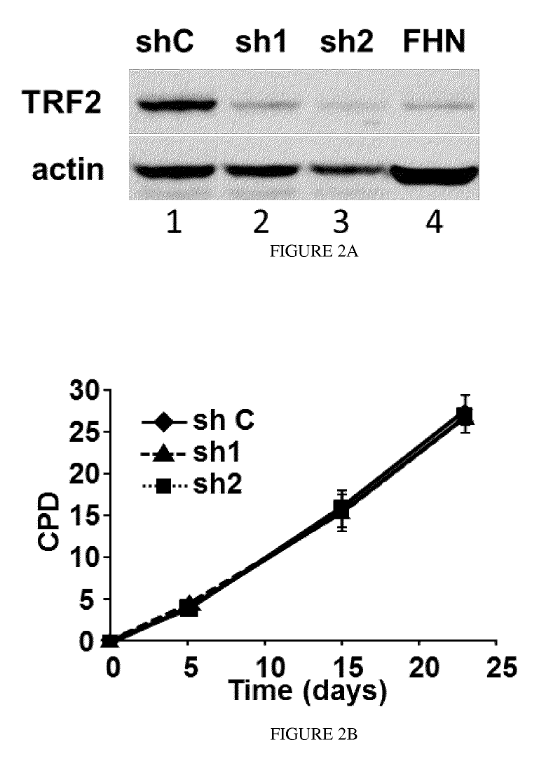

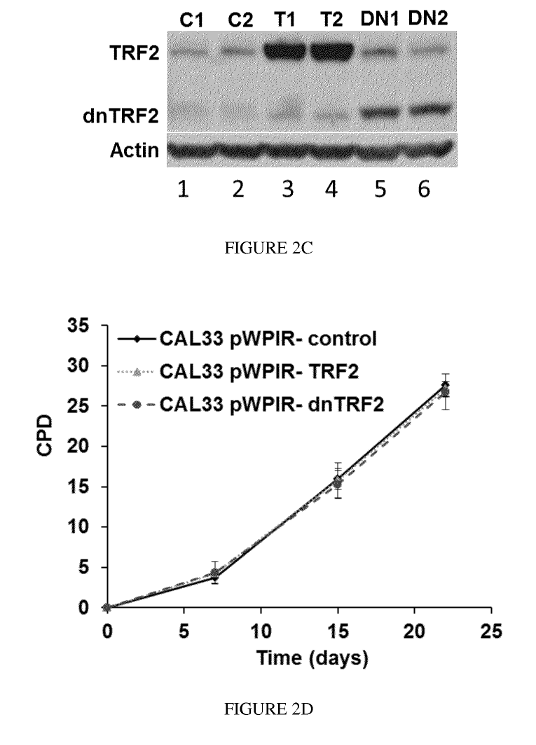

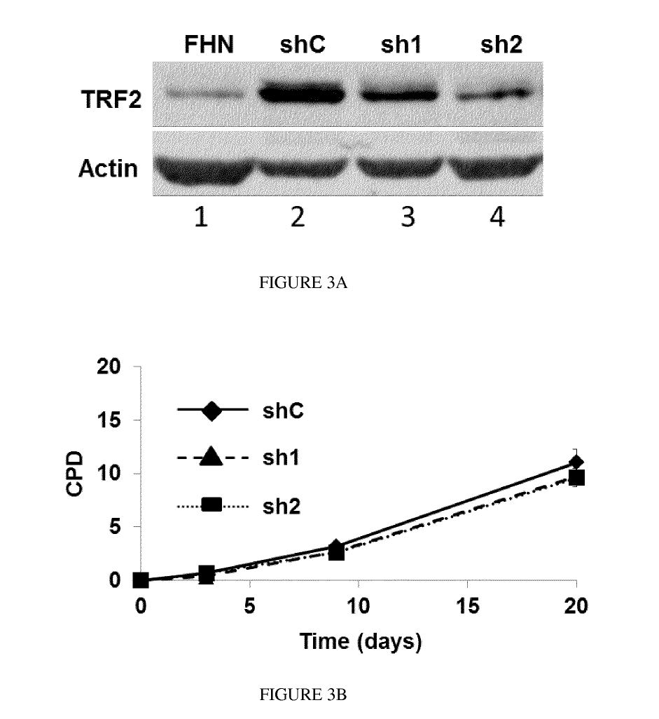

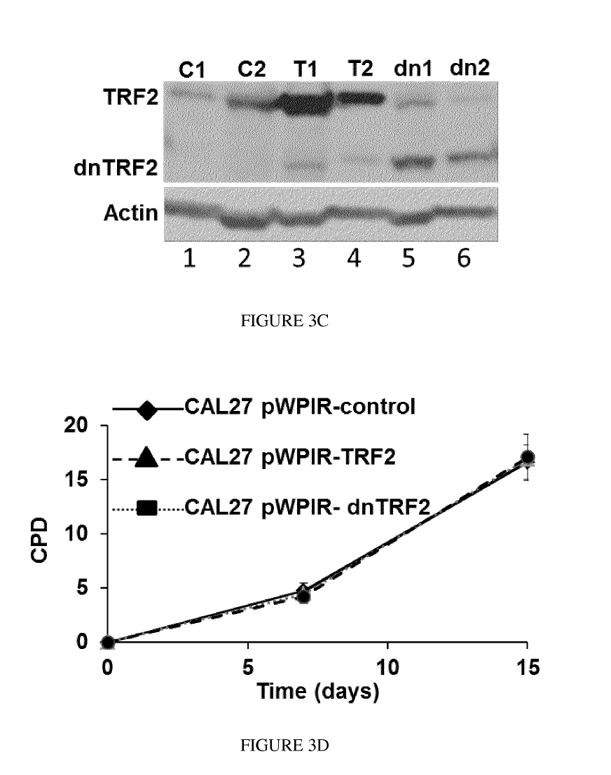

Because TRF2 expression in OSCC cells appears to be a prognostic marker, OSCC cell lines in vitro were next characterized. CAL33 cells showed significant higher TRF2 expression compared to normal human fibroblasts. Proliferative and invasive capacities of CAL33 cells knock-down for TRF2, or over-expressing a wild-type or a dominant negative form of TRF2 were then assessed (FIG. 2B). Three different shRNA were tested to compare their efficacy in knocking-down TRF2 in CAL33 cells and the shRNA #2 was the most efficient (FIG. 2A). No manipulations of TRF2 expression/activity influenced the proliferative and invasive capacities of CAL33 cells (FIGS. 2B and 2D). Equivalent results were obtained for CAL27 cells (FIGS. 3A, 3B, 3C and 3D). No difference in DNA damage attested by the number of 53BP1 foci was observed.

2.3—TRF2 Down-Regulation Modifies the Tumor Cell Secretome

The above results were apparently discrepant with the observation on patients' samples. However, they suggested that TRF2 expression may influence expression of specific factors that will act in a paracrine manner on cells of the microenvironment.

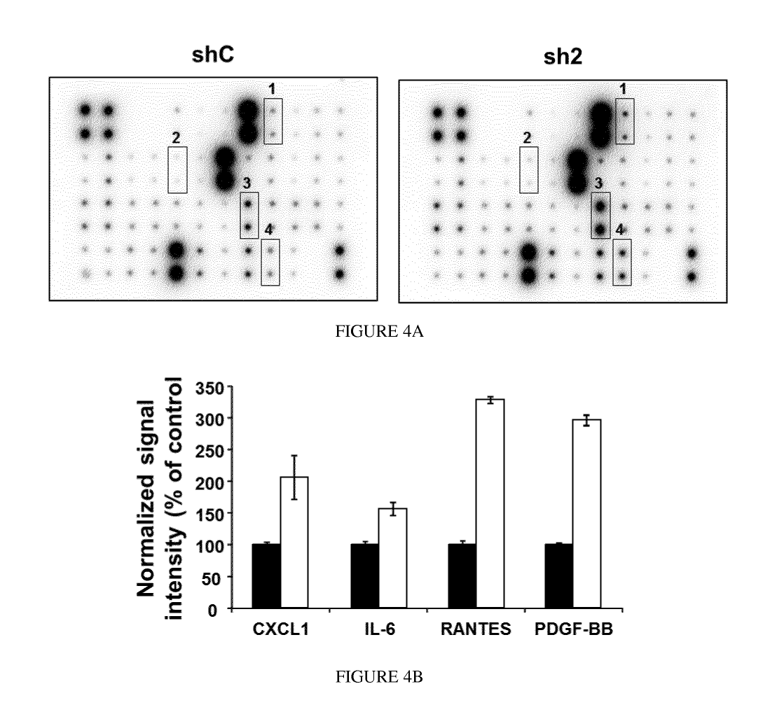

Therefore, cytokines expression in the supernatants of CAL33 cells with or without TRF2 knock-down were measured to verify the hypothesis that TRF2 expression influenced the secretome of the OSCC cells (FIGS. 4A and 4B). TRF2 knock-down in CAL33 resulted in the induction of CXCL1, CXCL8, CXCL9, CXCL10, interleukin 6 (IL6), PDGF-BB and RANTES and a decrease in VEGF expression (see Table 3).

TABLE 3
Analysis of pro-angiogenic/pro-inflammatory genes induced by TRF2
silencing in CAL 33 cells.

| CAL 33 | shC | sh1 | sh2 |
|---|---|---|---|
| 36B4 | 100 | 100 | 100 |
| m- | 100 | 100 | 100 |
| RPLP0 | | | |
| GAPDH | 100 | 100 | 100 |
| TRF2 | 100 | 70 | 40 |
| CXCL1 | 100 | 380(***) | 370(***) |
| CXCL7 | 100 | 110 | 120(*) |
| CXCL8 | 100 | 350(***) | 1100(***) |
| CXCL9 | 100 | 210(**) | 600(***) |
| IL6 | 100 | 400(***) | 1000(***) |
| PDGF- | 100 | 150(*) | 190(***) |
| BB | | | |
| RANTES | 100 | 270(***) | 190(***) |

The percentage expression of the different genes evaluated by qPCR is shown.
For the measured genes, the reference values (100) correspond to the content of a given gene in shC cells. The statistically significant differences are shown.
*p < 0.05: **p < 0.01: ***p < 0.001.

The cytokines genes inductions were almost identical in another cell line (CAL 27).

These results suggest that TRF2 served as a gene expression regulator independently of its telomere stabilizing effects.

2.4—TRF2 Knock-Down Decreased the Growth of OSCC Xenografts in Mice

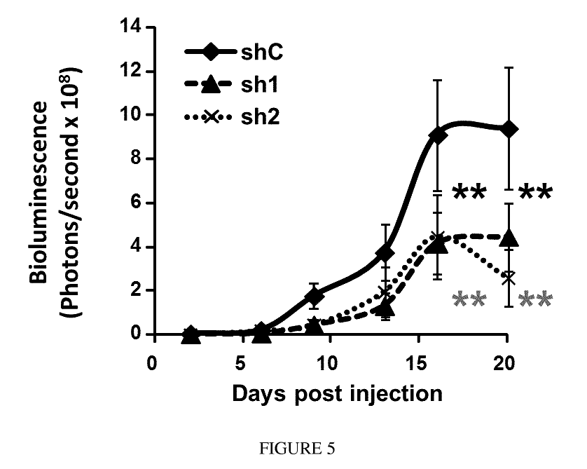

In order to assess the hypothesis that TRF2 expression in CAL33 cells was associated with tumor aggressiveness, CAL33 expressing either control or two independent TRF2-directed shRNA were injected in nude mice. Tumors with TRF2 knock-down were smaller, circular and with decreased invasive properties attested by luminescence limited to a zone around the injection site (FIG. 5). The smallest tumors were associated with the highest TRF2 knock-down (group shTRF2 #2).

2.5—TRF2 Knock-Down Prevents Blood Vessels Organization and Favors Fibrosis, Inflammation and Tumor Necrosis

To further understand how TRF2 expression modulated tumor growth, tumor sections were analyzed. Compared to control tumors, shTRF2 tumors were characterized by important necrotic zones (7.5% versus 26%, p=0.018) and thinner collagen layer around vessels (37 μm versus 7 μm, p=0.001). Moreover, inflammatory and red blood cells extravasation was observed around vessels in knocked-down TRF2 tumor sections suggesting acute inflammation and disorganized vascular network. Then, tumor sections were monitored for vascularization with CD31 (endothelial cells)/alpha-SMA (pericytes) labeling. Control tumors were characterized by a high blood vessel density with coverage of vessels with alpha-SMA-labeled cells. The vascular network of shTRF2 tumors was more anarchic and characterized by an absence of CD31/alpha-SMA co-labeling. Dispersed CD31 and alpha-SMA-positive cells were observed. The number of alpha-SMA-positive cells was characteristic of fibrotic zones.

2.6—CAL33 Cells Knocked-Down for TRF2 are More Sensitive to Erlotinib/Tarceva and Cetuximab/Erbitux

OSCC are characterized by over-expression of the EGFR receptor and subsequent activation of down-stream signaling pathways in particular the ERK/MAP Kinase and PI3 Kinase/AKT pathways which leads to abnormal proliferation (Bozec A et al. Ann Oncol 2009; 20(10):1703-7). Subsequently anti-EGFR therapy and in particular cetuximab are specifically used for patients with a poor performance status (Petrelli F et al. Oral Oncol 2014; 50(11):1041-8; Peyrade F et al. Oral Oncol 2013; 49(6):482-91).

The hypothesis that TRF2 expression may influence the response to major inhibitors of the EGFR in particular the tyrosine kinase inhibitor of the EGFR, erlotinib/Tarceva or EGFR targeted antibodies cetuximab/Erbitux was assessed. Erlotinib at a dose superior to the IC50 (5.4 μmol/L) efficiently inhibited EGFR activity and subsequently down-stream signaling pathways (ERK and AKT activity) CAL33 cells (Quesnelle K M et al. Cancer Biol Ther 2012; 13(10):935-45).

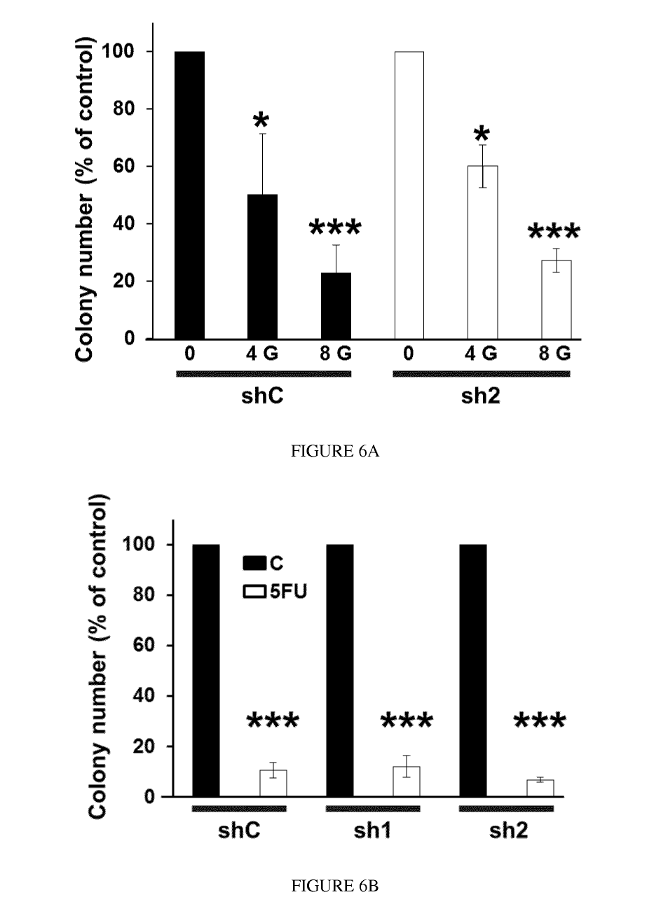

The knock-down of TRF2 has no influence on the efficacy of radiotherapy (FIG. 6A) or 5 FU (FIG. 6B), which are two therapeutic strategies for treating OSCC.

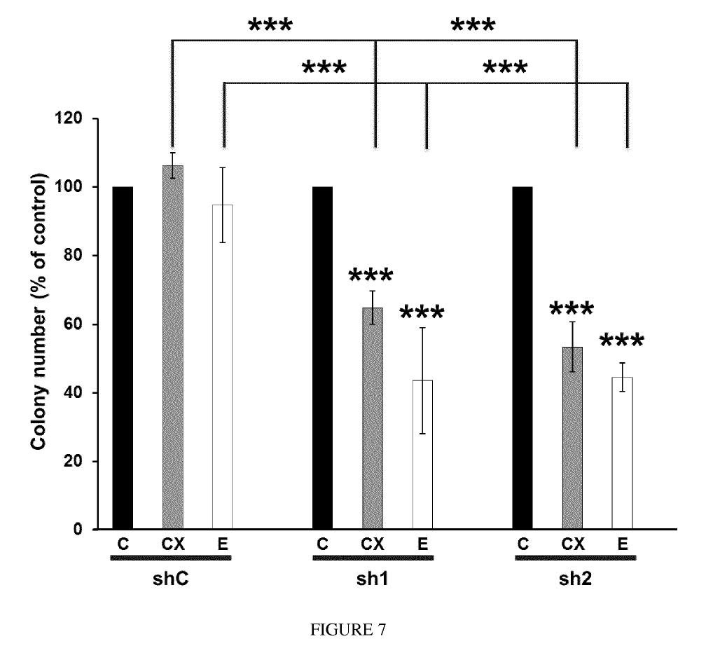

By contrast, knocking-down of TRF2 increased drastically the efficacy of erlotinib (0.1 μmol/L) and cetuximab (6 nmol/L) in CAL33 cells used at doses largely inferior to the IC50 for these two drugs that are respectively 5.4 μmol/L (Quesnelle K M et al. Cancer Biol Ther 2012; 13(10):935-45) and 30 nmol/L (Rebucci M et al. Int J Oncol 2011; 38(1):189-200) (40% and 60% inhibition for sh1 and 50% and 60% inhibition for sh2, FIG. 7).

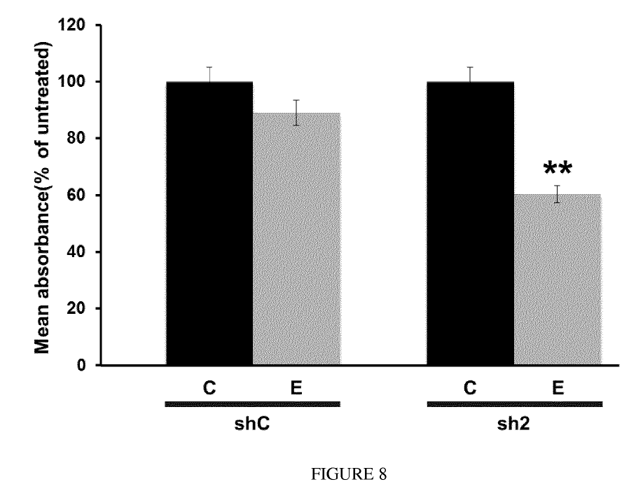

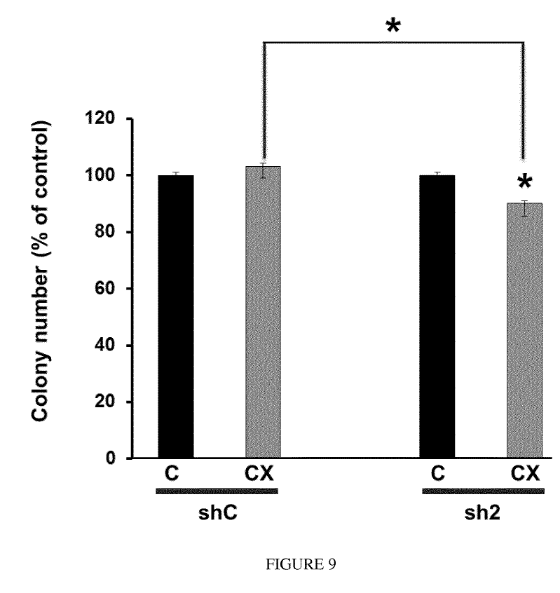

MTT tests confirmed that erlotinib inhibited CAL33 proliferation when TRF2 was knocked-down with sh2 (FIG. 8). The knock-down of TRF2 increased the efficacy of cetuximab in CAL 27 (FIG. 9). These results suggested that TRF2 expression may constitute a relevant predictive marker of anti-EGFR treatments in OSCC.

3—Discussion

Numerous articles describe the role of TRF2 in cancers but no published articles describe the role of TRF2 in OSCC.

An easy TRF2 reading score is hereby provided for pathologists, by immunohistochemistry with a commercially available monoclonal anti-TRF2 antibody. Moreover, TRF2 expression was easy to determine on the diagnostic biopsy which is always performed to confirm the presence of a tumor. Such TRF2 expression has to be determined in a prospective study to confirm its relevance as a prognostic marker.

In vitro assays showed that OSCC cells expressing high levels of TRF2 did not have any advantages in terms of proliferation, migration and invasion which confirmed previous results (Biroccio A et al. Nat Cell Biol 2013; 15(7):818-28). However, TRF2 was shown to modulate tumor microenvironment through RAS-dependent expression of IL6 (Biroccio A et al. Nat Cell Biol 2013; 15(7):818-28). In the experimental model herein over-expression/activation of EGFR leads to activation of the RAS pathway which explains that the tested cells express high basal levels of IL6. Biroccio et al described that IL6 compensate for the loss of TRF2 in the presence of active RAS in order to sustain cell proliferation.

The different genes induced by TRF2 down-regulation are implicated in inflammatory processes and in the angiogenesis balance and their induction may seem discrepant considering the in vivo effect associated with decreased TRF2 expression. Except for CXCL9 and CXCL10 that have anti-angiogenic properties and PDGF-BB that inhibits the growth of angiogenesis-dependent tumors because of increased pericyte coverage of blood vessels, the other induced cytokines are considered as markers of poor prognosis (RANTES, IL6, IL8, GRO-alpha) (Aldinucci D, Colombatti A. Mediators Inflamm 2014; 2014:292376; Mauer J et al. Trends Immunol 2015; 36(2):92-101; Zarogoulidis P et al. Cancer Invest 2014; 32(5):197-205; Verbeke H et al. Cytokine Growth Factor Rev 2011; 22(5-6):345-58).

Although the experiments were performed in nude mice, the presence of natural killers and macrophages may still participate in anti-tumor program. Therefore, tumors corresponding to TRF2 expression 0/1+ may induce expression of cytokines that enhance anti-tumor immune response, RANTES appearing as a beneficial factor to be used as an adjuvant for cancer immunotherapy (Lapteva N, Huang X F. Expert Opin Biol Ther 2010; 10(5):725-33). IL6 is also considered as a pro-tumor factor. However, IL6 can promote B-cell differentiation (B cells are present in nude mice) which is thought to prevent tumor growth. Moreover, GRO-alpha/CXCL1 and IL8/CXCL8 are major chemo attractants for leukocytes that play a key role for immune depletion of cancer cells. Hence, tumor cells with low TRF2 levels expressed more chemo-attractants for immune cells and indirectly slowed-down tumor growth.

The experimental data herein also suggested that TRF2 may represent a relevant predictive marker for treatment response to targeted therapies cetuximab/Erbitux and erlotinib/Tarceva. Cetuximab is only used for patient with poor performance status because it is less aggressive than the generally used platinum salts. However, sensitivity to cetuximab can be altered by mutation of RAS, overexpression of HER2 or activation of the PI3 kinase activity. Erlotinib/Tarceva was used for patients with head and neck tumors but only 50% of them have an objective response to the treatment (Agulnik M et al. J Clin Oncol 2007; 25(16):2184-90). The EGFR/pEGFR levels in skin biopsies were considered as surrogate markers but the authors suggested that additional markers are needed for a better evaluation of responders. Therefore TRF2 may be detected on the same skin biopsies to stratify the relevant patients to treat. A recent study described the detection of the EGFR/pEGFR ratio in tumors treated with erlotinib/Tarceva in a neo-adjuvant setting (Tsien C I et al. Head Neck 2013; 35(9):1323-30). These authors described tumor heterogeneity in terms of response to the drug and activity of EGFR. These results suggest that a more accurate determination of responders is required. Erlotinib/Tarceva is intensively used to treat patients with lung cancer but the patients must be tested for the presence of specific mutations of the EGFR that predict treatment efficacy. The mutations encountered in lung cancers were not observed in head and neck tumors but other mutations were reported. The value of these mutations as predictive marker of response has not been validated for the moment but may be concomitantly analyzed with TRF2 levels. In conclusion, it is hereby provided a valuable tool for the determination of individuals at risk of recurrence and stratify patients that can benefit of anti-EGFR targeted therapies.

### REFERENCES

Agulnik M, da Cunha Santos G, Hedley D, et al. Predictive and pharmacodynamic biomarker studies in tumor and skin tissue samples of patients with recurrent or metastatic squamous cell carcinoma of the head and neck treated with erlotinib. J Clin Oncol 2007; 25(16):2184-90.

Aldinucci D, Colombatti A. The inflammatory chemokine CCL5 and cancer progression. Mediators Inflamm 2014; 2014:292376.

Ausubel F M et al. Current Protocols In Molecular Biology; John Wiley and Sons, NY, 1987.

Bellon M et al. Increased expression of telomere length regulating factors TRF1, TRF2 and TIN2 in patients with adult T-cell leukemia. Int J Cancer 2006; 119(9):2090-7.

Biroccio A, Cherfils-Vicini J, Augereau A, et al. TRF2 inhibits a cell-extrinsic pathway through which natural killer cells eliminate cancer cells. Nat Cell Biol 2013; 15(7):818-28.

Boettcher M, McManus M T. Choosing the Right Tool for the Job: RNAi, TALEN, or CRISPR. Mol Cell. 2015 May 21; 58(4):575-585.

Bozec A, Sudaka A, Toussan N, et al. Combination of sunitinib, cetuximab and irradiation in an orthotopic head and neck cancer model. Ann Oncol 2009; 20(10):1703-7.

Brauer A U, Savaskan N E, Kole M H, et al. Molecular and functional analysis of hyperpolarization-activated pacemaker channels in the hippocampus after entorhinal cortex lesion. FASEB J, 2001; 15, 2689-2701.

Chuang H C, Chen C H, Huang C C et al. Reduced expression of TRF1 is associated with tumor progression and poor prognosis in oral squamous cell carcinoma. Exp Ther Med. 2011; 2(1):63-67.

Da-Silva N et al. Altered mRNA expression of telomere binding proteins (TPP1, POT1, RAP1, TRF1 and TRF2) in ulcerative colitis and Crohn's disease. Dig Liver Dis 2010; 42(8):544-8.

Diehl M C, Idowu M O, Kimmelshue K N, et al. Elevated TRF2 in advanced breast cancers with short telomeres. Breast Cancer Res Treat. 2011; 127(3):623-30.

Dong W et al. Sp1 upregulates expression of TRF2 and TRF2 inhibition reduces tumorigenesis in human colorectal carcinoma cells. Cancer Biol Ther 2009; 8(22):2166-74.

Fujita et al. Positive feedback between p53 and TRF2 in telomere damage signaling and cellular senescence. Nat Cell Biol. 2010 December; 12(12): 1205-1212.

Gioanni J, Fischel J L, Lambert J C, et al. Two new human tumor cell lines derived from squamous cell carcinomas of the tongue: establishment, characterization and response to cytotoxic treatment. Eur J Cancer Clin Oncol 1988; 24(9):1445-55.

Grepin R et al. Acceleration of clear cell renal cell carcinoma growth in mice following bevacizumab/Avastin treatment: the role of CXCL cytokines. Oncogene 2012; 31(13):1683-94.

Harlow and Lane. Antibodies: A Laboratory Manual”, 2nd Ed; Cold Spring Harbor Laboratory Press, Cold Spring Harbor, N.Y., 1988.

Hoheisel J D. Microarray technology: beyond transcript profiling and genotype analysis. Nature Reviews, Genetics, 2006, 7(3):200-210.

Hu H et al. Expression of TRF1, TRF2, TIN2, TERT, KU70, and BRCA1 proteins is associated with telomere shortening and may contribute to multistage carcinogenesis of gastric cancer. J Cancer Res Clin Oncol 2010; 136(9):1407-14.

Klapper W et al. DNA damage transiently increases TRF2 mRNA expression and telomerase activity. Leukemia 2003; 17(10):2007-15.

Lapteva N, Huang X F. CCL5 as an adjuvant for cancer immunotherapy. Expert Opin Biol Ther 2010; 10(5):725-33.

Matsutani N et al. Expression of telomeric repeat binding factor 1 and 2 and TRF1-interacting nuclear protein 2 in human gastric carcinomas. Int J Oncol 2001; 19(3):507-12.

Mauer J, Denson J L, Bruning J C. Versatile functions for IL-6 in metabolism and cancer. Trends Immunol 2015; 36(2):92-101.

Munoz P et al. XPF nuclease-dependent telomere loss and increased DNA damage in mice overexpressing TRF2 result in premature aging and cancer. Nat Genet 2005; 37(10):1063-71.

Nakanishi K et al. Expression of mRNAs for telomeric repeat binding factor (TRF)-1 and TRF2 in atypical adenomatous hyperplasia and adenocarcinoma of the lung. Clin Cancer Res 2003; 9(3):1105-11.

Nekanti U, Dastidar S, Venugopal P, et al. Increased proliferation and analysis of differential gene expression in human Wharton's jelly-derived mesenchymal stromal cells under hypoxia. Int J Biol Sci 2010; 6(5):499-512.

Oh B K et al. Up-regulation of telomere-binding proteins, TRF1, TRF2, and TIN2 is related to telomere shortening during human multistep hepatocarcinogenesis. Am J Pathol 2005; 166(1):73-80.

Padhi S, Saha A, Kar M, Ghosh C et al. Clinico-Pathological Correlation of β-Catenin and Telomere Dysfunction in Head and Neck Squamous Cell Carcinoma Patients. J Cancer. 2015; 6(2):192-202.

Perri F, Muto P, Aversa C, et al. Integrated therapeutic approaches in head and neck cancer: the importance of multidisciplinary team management. Anticancer Agents Med Chem 2013; 13(6):834-43.

Petrelli F, Coinu A, Riboldi V, et al. Concomitant platinum-based chemotherapy or cetuximab with radiotherapy for locally advanced head and neck cancer: a systematic review and meta-analysis of published studies. Oral Oncol 2014; 50(11):1041-8.

Peyrade F, Cupissol D, Geoffrois L, et al. Systemic treatment and medical management of metastatic squamous cell carcinoma of the head and neck: review of the literature and proposal for management changes. Oral Oncol 2013; 49(6):482-91.

Quesnelle K M, Wheeler S E, Ratay M K, et al. Preclinical modeling of EGFR inhibitor resistance in head and neck cancer. Cancer Biol Ther 2012; 13(10):935-45.

Rebucci M, Peixoto P, Dewitte A, et al. Mechanisms underlying resistance to cetuximab in the HNSCC cell line: role of AKT inhibition in bypassing this resistance. Int J Oncol 2011; 38(1):189-200.

Sainger R N, Telang S D, Shukla S N, Patel P S. Clinical significance of telomere length and associated proteins in oral cancer. Biomark Insights. 2007 Feb. 14; 2:9-19.

Tsien C I, Nyati M K, Ahsan A, et al. Effect of erlotinib on epidermal growth factor receptor and downstream signaling in oral cavity squamous cell carcinoma. Head Neck 2013; 35(9):1323-30.

Tuerk C and Gold L. Systematic evolution of ligands by exponential enrichment: RNA ligands to bacteriophage T4 DNA polymerase. Science, 1990; vol. 249(4968), 505-10.

Ul Ain Q, Chung J Y, Kim Y H. Current and future delivery systems for engineered nucleases: ZFN, TALEN and RGEN. J Control Release. 2015 May 10; 205:120-7.

Verbeke H, Struyf S, Laureys G, et al. The expression and role of CXC chemokines in colorectal cancer. Cytokine Growth Factor Rev 2011; 22(5-6):345-58.

Wilkie G. S. and Schirmer E. C. Chapter 2 Purification of Nuclei and Preparation of Nuclear Envelopes from Skeletal Muscle. Hancock (ed.) The Nucleus: Volume 1: Nuclei and sub-nuclear components (copyright Humana Press 2008) ISBN: 978-1-4939-1679-5 (Print) 978-1-4939-1680-1 (Online).

Wright D A, Li T, Yang B, Spalding M H. TALEN-mediated genome editing: prospects and perspectives. Biochem J. 2014 Aug. 15; 462(1):15-24.

Zarogoulidis P, Katsikogianni F, Tsiouda T, et al. Interleukin-8 and interleukin-17 for cancer. Cancer Invest 2014; 32(5):197-205.

1. An in vitro method for predicting a likelihood of an individual having a cancer to efficiently respond to an anti-cancer treatment, said method comprising the steps of:
a) measuring the level of nuclear expression of TRF2 in a sample obtained from said individual,
b) comparing the level obtained in step a) to a reference value, and
c) determining the predicted likelihood of said individual to efficiently respond to said anti-cancer treatment from the comparison performed in step b).

2. The in vitro method according to claim 1, wherein a level of nuclear expression of TRF2 as measured in step a) lower than a reference value measured in a sample of a non-cancerous tissue is indicative of a good predicted likelihood of said individual having a cancer to efficiently respond to said anti-cancer treatment.

3. The in vitro method according to claim 1, wherein said anti-cancer treatment comprises the administration of an antagonist of EGFR, preferably selected in a group comprising an EGFR tyrosine kinase inhibitor and a compound that specifically binds to EGFR.

4. The in vitro method according to claim 1, wherein said cancer is an oral squamous cell carcinoma.

5. An in vitro method for predicting an outcome for an individual having an oral squamous cell carcinoma, said method comprising the steps of:
a) measuring a level of nuclear expression of TRF2 in a sample obtained from said individual,
b) comparing a level obtained in step (a) to a reference value, and
c) determining a prognostic of said individual from said comparison performed in step b).

6. The in vitro method according to claim 5, wherein a level of nuclear expression of TRF2 as measured in step a) lower than a reference value measured in a sample of a non-cancerous tissue is indicative of a good outcome for said individual.

7. A method for the treatment of an individual having an oral squamous cell carcinoma comprising a step of administering an inhibitor of the TRF2 gene expression to the said individual.

8. The method according to claim 7, wherein said inhibitor of the TRF2 gene expression is selected from an antisense DNA, an antisense RNA, a double stranded RNA, a mi RNA, a siRNA, a shRNA, an aptamer specifically binding to the mRNA encoded by a TRF2 gene and a non-specific inhibitor of TRF2 gene expression.

9. A kit for use in the treatment of an individual having an oral squamous cell carcinoma comprising:
an inhibitor of the TRF2 gene expression in a physiologically acceptable excipient, and,
an anti-cancer compound in a physiologically acceptable excipient.

10. The kit for its use according to claim 9, wherein said inhibitor of the TRF2 gene expression is selected from an antisense DNA, an antisense RNA, a double stranded RNA, a miRNA, a siRNA, a shRNA, an aptamer specifically binding to the mRNA encoded by a TRF2 gene and a non-specific inhibitor of TRF2 gene expression.

11. The kit for its use according to claim 9, wherein said anti-cancer compound is an EGFR antagonist.

12. The kit according to claim 9, wherein said anti-cancer compound is an EGFR antagonist and wherein said EGFR antagonist is selected in a group comprising an EGFR tyrosine kinase inhibitor and a compound that specifically binds to EGFR, preferably to the extracellular domain of EGFR.

13. The kit according to claim 11, wherein said anti-cancer compound is an EGFR antagonist and wherein said EGFR antagonist is selected in a group comprising Erlotinib, Gefitinib, Lapatinib, Cetuximab, Panitumumab, Nimotuzumab, Matuzumab and Zalutumumab.

14. A method for predicting an outcome for an individual having an oral squamous cell carcinoma comprising a step of determining ex vivo or in vitro a level of nuclear expression of TRF2 as a biomarker.

15. A method for predicting a likelihood of an individual having an oral squamous cell carcinoma to efficiently respond to an anti-cancer treatment comprising a step of determining ex vivo or in vitro a level of nuclear expression of TRF2 as a biomarker.

16. A method for determining a severity of an oral squamous cell carcinoma in an individual comprising a step of determining ex vivo or in vitro a level of nuclear expression of TRF2 as a biomarker.
